(19) United States
(12) Patent Application Publication
(10) Pub. No.: US 2024/0066175 A1
(43) Pub. Date: Feb. 29, 2024

(54) HYDROXYL ION GENERATOR APPARATUSES FOR CEILING MOUNT OR WALK THROUGH

(71) Applicant: TURNTEK, LLC, Greenwood, IN (US)

(72) Inventors: Robert Edward Turner, Indianapolis, IN (US); Brian Whitney Williams, Indianapolis, IN (US); Gregory A. Cox, Greenwood, IN (US); James Spencer Alonso, Indianapolis, IN (US); Matthew M. Cox, Avon, IN (US)

(21) Appl. No.: 18/237,569

(22) Filed: Aug. 24, 2023

Related U.S. Application Data
(60) Provisional application No. 63/373,742, filed on Aug. 29, 2022.
(60) Provisional application No. 63/383,541, filed on Nov. 14, 2022.

(51) Int. Cl. A61L 9/14

(52) U.S. Cl. CPC A61L 9/14; A61L 2209/111; A61L 2209/134

(57) ABSTRACT

The invention describes a method and a walk through apparatus to treat surfaces with hydroxyl ions to reduce the viability and/or kill pathogens.

### CROSS-REFERENCE TO RELATED APPLICATIONS

This application claims priority to U.S. Patent Application Ser. No. 63/373,742, filed Aug. 29, 2022 and U.S. Patent Application Ser. No. 63/383,541, filed Nov. 14, 2022, which are each herein incorporated by reference.

### FIELD OF THE INVENTION

The invention relates generally to an apparatus of sufficient dimensions such that an individual can pass through the apparatus wherein the apparatus generates sufficient hydroxyl ion content to treat and/or kill pathogens, such as allergens, pollen, protozoa, fungi, molds, viruses, bacteria, etc. on the individual or surfaces of objects passed through the apparatus. In another aspect, the invention relates generally to an apparatus that can be used in enclosed areas, such as office spaces, restaurants, bars, storage rooms, warehouses, classrooms, etc., by filtering air, passing the filtered air through a hydroxyl ion generator and distributing the hydroxyl ions into the enclosed area to reduce or eliminate pathogens, such as allergens, pollen, protozoa, fungi, molds, viruses, bacteria, etc. in the enclosed space and/or on the surfaces within the enclosed space.

### BACKGROUND OF THE INVENTION

Coronaviruses are a group of related RNA viruses that affect mammals and birds. In humans and birds, such viruses can cause respiratory tract infections that range from mild to lethal. Mild illnesses in humans include some cases of the common cold (which is also caused by other viruses, predominantly rhinoviruses), while more lethal varieties can cause SARS, MERS, and COVID-19 as well as COVID-19 variants. A few vaccines are currently being marketed to treat COVID-19, however, what is termed the “delta” variant appears to be more virulent and is causing some breakthrough cases where the vaccine is not completely effective.

In particular, COVID-19 and variants thereof, such as the delta and mu variants, also referred to as SARS-CoV-2 and the variants thereof, is thought to spread mainly from person to person, mainly through respiratory droplets produced when an infected person coughs or sneezes. These droplets can land in the mouths or noses of people who are nearby or possibly be inhaled into the lungs. It is thought that spread is more likely when people are in close contact with one another (within about 6 feet). It may be possible that a person can get COVID-19 or a variant by touching a surface or object that has the virus on it and then touching their own mouth, nose, or possibly their eyes, however, there is no definitive answer at this time.

An important issue associated with coronaviruses, such as COVID-19 and variants, is how to control the spread of the virus. This aspect is especially important to consider when individuals travel about the world via public transportation, airplanes, trains and the like. Additionally, questions exist as to how to control the spread of the virus in settings where there are often large gatherings of individuals such as schools, restaurants, office buildings, hospitals, clinics, emergency rooms and the like.

Therefore, a need exists for an efficient method and apparatus that can minimize and or eliminate airborne or surface deposited viruses.

### BRIEF SUMMARY OF THE INVENTION

The present embodiments surprisingly provides methods and apparatus' to subject an object, such as an individual, to an atmosphere containing hydroxyl ions to reduce or eliminate unwanted pathogens, such as protozoa, fungi, molds, bacteria and/or viruses, such as corona viruses.

The present embodiments also provide methods and apparatus' to reduce or eliminate unwanted constituents in the air such as volatile organic components or smoke.

In one aspect, the embodiments described herein provide a portal comprising a plurality of orifices, e.g., nozzles, arranged in an array and positioned on at least one of a first and/or a second opposing side of the portal and a hydroxyl ion generator in communication with the portal and the plurality of orifices, such as nozzles. Alternatively, the orifice can be an opening within the portal that can transmit the hydroxyl ions away from the apparatus. In one embodiment, the orifice can be a screen. Hydroxyl ions are transmitted through the orifices to emit the hydroxyl ions into the area formed by the portal. An object, such as a person, can pass through the portal and be treated with hydroxyl ions to reduce or eliminate unwanted pathogens, such as protozoa, fungi, molds, bacteria and/or viruses. The apparatus and method provide a safe and effective approach to reducing and/or eliminating harmful pathogens. Hydroxyl ions are safe for use with humans/mammals and will not harm an individual or surface(s). This approach as the advantages of being very efficient in the destruction of unwanted pathogens, such as protozoa, fungi, molds, bacteria and/or viruses, short contact times for effectiveness, the ability to treat a surface without harm to the surface and, for example, does not harm plant life/vegetation.

In another aspect, the embodiments relate generally to an apparatus that can be used in enclosed areas, such as office spaces, restaurants, bars, storage rooms, warehouses, classrooms, etc. by filtering air, passing the filtered air through a hydroxyl ion and distributing the hydroxyl ions into the enclosed area to reduce the concentration and/or kill pathogens, such as allergens, pollen, protozoa, fungi, molds, viruses, bacteria, etc. in the enclosed space and/or on the surfaces within the enclosed space.

In yet another aspect of the disclosed embodiments, an apparatus includes a hydroxyl ion generator configured to emit hydroxyl ions into a surrounding area. An atomizer system is configured to generate water droplets from water retained in a cavity. An atomizer is positioned in the cavity and is configured to be submerged by the water in the cavity. The atomizer generates the water droplets. A fan is positioned adjacent the atomizer system. The fan is configured to direct the water droplets from the atomizer system into the hydroxyl ion generator.

In some embodiments, the apparatus may include a portal having at least one opening. The hydroxyl ions may be transmitted through the at least one opening to emit the hydroxyl ions into an area formed by the portal. The hydroxyl ion generator may include a perforated mesh coated with titanium dioxide and configured to collect the water droplets. A UV light generator may direct a spectrum of light from about 320 nm to about 385 nm onto the perforated mesh. The water source may be at least one of a continuous water source and a container. The atomizer system may provide at least 60% humidity to the hydroxyl ion generator. A hydroxyl concentration of the hydroxyl ions may be at a level that reduces or eliminates one or more pathogens upon exposure to the hydroxyl ions.

Optionally, the apparatus may include a control system having a humidity sensor to monitor a humidity discharged by the apparatus. A temperature sensor may monitor a temperature of the hydroxyl ions. A flow sensor may monitor a flow rate of the hydroxyl ions. The control system may adjust the atomizer based on at least one of the humidity as measured by the humidity sensor, the temperature as measured by the temperature sensor, and the flow rate as measured by the flow sensor. The control system may adjust the hydroxyl ion generator based on at least one of the humidity as measured by the humidity sensor, the temperature as measured by the temperature sensor, and the flow rate as measured by the flow sensor. A UV light generator may direct a spectrum of light from about 320 nm to about 385 nm onto the water droplets. The control system may adjust the atomizer based on at least one of the temperature as measured by the temperature sensor, and the flow rate as measured by the flow sensor.

It may be desired that the apparatus includes an ozone sensor and the apparatus may be inactivated if a predetermined level of ozone is detected in an area around the apparatus. The apparatus may include a carbon dioxide sensor and the apparatus may be inactivated if a predetermined level of carbon dioxide is detected in the area around the apparatus. The apparatus may include a carbon monoxide sensor and the apparatus may be inactivated if a predetermined level of carbon monoxide is detected in the area around the apparatus.

In some embodiments, the apparatus may include a plurality of apparatuses including a master apparatus and at least one slave apparatus that is controlled by the master apparatus. Each of the master apparatus and the at least one slave apparatus may include a wireless transceiver. The master apparatus and the at least one slave apparatus may communicate through the wireless transceiver. The master apparatus and the at least one slave apparatus may be remotely controllable by a remote device through the wireless transceiver.

It may be contemplated that the atomizer system may include a water inlet configured to couple to a water source to dispense water into the cavity. The atomizer system further may include a sensor configured to detect an amount of water in the cavity. A flow of water into the cavity is controlled based on the amount of water detected in the cavity. The atomizer system may include an air intake configured to direct air into the cavity. An air outlet may be configured to direct air containing the water droplets out of the cavity. The air outlet may be positioned adjacent the fan. The fan may be configured to direct the air containing the water droplets into the hydroxyl ion generator.

In some embodiments, the apparatus may include a portable cart and the apparatus may be positioned on the portable cart. The apparatus may be installed in at least one of a floor, a wall, and a ceiling.

While multiple embodiments are disclosed, still other embodiments of the present invention will become apparent to those skilled in the art from the following detailed description. As will be apparent, the invention is capable of modifications in various obvious aspects, all without departing from the spirit and scope of the present invention. Accordingly, the detailed descriptions are to be regarded as illustrative in nature and not restrictive.

### BRIEF DESCRIPTION OF THE DRAWINGS

FIG. 1 depicts a cut away view of a portal apparatus system 10.

FIG. 2 depicts an exterior view of hollow side panel 20 of portal apparatus system 10 showing air filter 120.

FIG. 3 depicts an interior view of hollow side panel 20 of portal apparatus system 10 with orifices 130 shown.

FIG. 4 depicts an optional top portion 140 of portal apparatus system 10 with orifices 130 (e.g., nozzles).

FIG. 5 depicts a side view of portal apparatus system 10 showing side panels 20, the inflection point for radius 35 to optional top portion 140 and orifices 130.

FIG. 6 depicts a circular hydroxyl generating ion assembly, wherein the panel is formed as a single integral unit.

FIG. 7 depicts a circular hydroxyl generating ion assembly, wherein the panel is formed from multiple pieces.

FIG. 8 depicts a trapezoidal hydroxyl generating ion assembly.

FIG. 9 depicts a circular hydroxyl generating ion assembly, wherein the assembly is formed from at least one tube.

FIG. 10 depicts a top and/or bottom view of an apparatus suitable for use in an enclosed area via a ceiling mount or as a stand-alone unit.

FIG. 11 depicts the enclosed area unit apparatus hydroxyl generating ion components/assembly.

FIG. 12 depicts a hydroxyl generating ion assembly positioned in a top of a door frame.

FIG. 13 depicts a hydroxyl generating ion assembly positioned on a floor.

FIG. 14 depicts a hydroxyl generating ion assembly embedded in a floor.

FIG. 15 depicts a cut-away of a directional airflow apparatus having a single UVA bulb and fan.

FIG. 16 is a perspective view of the apparatus shown in FIG. 15.

FIG. 17 is an external view of the apparatus shown in FIG. 15.

FIG. 18 is an alternative embodiment of the apparatus shown in FIG. 15.

FIG. 19 depicts a cut-away of a directional airflow apparatus having a double UVA bulb and fan.

FIG. 20 is a perspective view of the apparatus shown in FIG. 19.

FIG. 21 is an external view of the apparatus shown in FIG. 19.

FIG. 22 is a front view of a water channel that is configured for operation in any one of the apparatuses shown in FIGS. 15-21.

FIG. 23 is a schematic view of a control system configured for any of the embodiments described herein.

FIG. 24 is a schematic view of a plurality of directional airflow apparatuses including a master unit and at least one slave unit.

FIG. 25 is a graph illustrating the data from 3 separate trials conducted on the apparatus described herein in a 16 m³ chamber over the course of 60 minutes.

FIG. 26 is a front view of a portable directional airflow apparatus.

FIG. 27 is a rear cut away view of the portable directional airflow apparatus shown in FIG. 26 and showing the interior components of the directional airflow apparatus.

FIG. 28 depicts the water channel shown in FIG. 27, wherein the water channel is suitable for use with any of the directional airflow apparatuses described herein.

FIG. 29 depicts the water channel shown in FIG. 28 having components positioned therein.

FIG. 30 depicts a water tank of the directional airflow apparatus shown in FIG. 26.

FIG. 31 depicts a valve system for use with the water tank shown in FIG. 30.

FIG. 32 depicts a cover for the fan shown in FIG. 27, wherein the cover retains an atomizer.

FIG. 33 depicts an embodiment of an atomizer system that is usable with the systems and apparatuses described herein.

FIG. 34 depicts another view of the atomizer system shown in FIG. 33.

FIG. 35 depicts an interior view of the atomizer system shown in FIG. 33.

FIG. 36 depicts the atomizer shown in FIG. 33 installed in an embodiment of a directional airflow apparatus.

FIG. 37 depicts an atomizer in accordance with an embodiment.

FIG. 38 depicts an interior view of an atomizer system formed in accordance with another embodiment.

FIG. 39 depicts a closed view of the atomizer system shown in FIG. 38.

FIG. 40 depicts an exemplary float sensor utilized with the atomizer system shown in FIG. 38.

FIG. 41 depicts an exemplary UV bulb utilized with any of the systems described herein.

FIG. 42 depicts an apparatus having a hydroxyl ion generator and incorporating the atomizer system shown in FIGS. 38-39 and the UV bulb shown in FIG. 41.

FIG. 43 depicts an embodiment of a directional airflow apparatus utilized in an exemplary test.

FIG. 44 depicts a stainless steel bioaerosol test chamber used for testing an embodiment of a directional airflow apparatus. The chamber is equipped with HEPA in/out filtration, multiple bioaerosol sampling ports, humidity and temperature control, and air pressure balancing.

FIG. 45 depicts a bioaerosol test chamber flow diagram. The chamber is equipped with bioaerosol induction, multiple bioaerosol sampling ports, particle size monitoring, internal mixing fans, and temperature and humidity controls.

FIG. 46 depicts a 6-jet collison nebulizer with glass and 304 stainless steel construction.

FIG. 47 depicts a SKC single stage biostage viable cascade impactor used for bacterial and spore sampling for select time points during bioaerosol trials. LOD is >0.01 cfu/L.

FIG. 48 depicts a TSI aerodynamic particle sizer (APS) model 3321 used to measure total particle concentration and particle size distribution of the challenge bioaerosol. Range 0.54-20.0 μm aerodynamic diameter, with 1 particle/L detection limits.

FIG. 49 depicts an FDA graphic demonstrating general resistance to disinfection for various microorganisms. FDA, guidance enforcement policy for sterilizers, disinfectant devices, and air purifiers during the Coronavirus Disease 2019 (COVID-19). SAR-CoV-2 (lipid or medium-Sized Virus), MS2 (non-lipid small virus).

FIG. 50 depicts an aerodynamic particle size distribution of RNA virus MS2 and DNA virus PhiX 174 in the test chamber. MMAD for both viral species averaged approximately 0.7 μm.

FIG. 51 depicts an aerodynamic particle size distribution of Aspergillus brasiliensis and Bacillus subtilis in the test chamber. MMAD for each species was approximately 1-3 μm.

FIG. 52 depicts an aerodynamic particle size distribution of Staph epidermidis and Klebsiella aerogenes in the bioaerosol test chamber. MMAD for each species was approximately 2.4-2.6 μm.

FIG. 53 depicts a test matrix for an embodiment of a directional airflow apparatus.

FIG. 54 depicts an approximate sampling timeline for an embodiment of a directional airflow apparatus.

FIG. 55 depicts net log reduction results for an embodiment of a directional airflow apparatus. Summary plot of averaged results data for all organisms.

FIG. 56 depicts executive summary data for all organisms. Averages for triplicate trials for each organism.

FIG. 57 depicts an MS2 graph illustrating the effect of hydroxyl radical production. Control data is based off of a single trial. The no-filter data consists of an average of two trials while the fan and filter data consist of an average of three trials.

FIG. 58 depicts an average LOG reduction for Bacillus subtillus trials with control trial data.

FIG. 59 depicts an average Net LOG reduction for Bacillus subtillus trials with control trial data.

FIG. 60 depicts an average LOG reduction for Aspergillus brasiliensis (previously A. niger) trials with control trial data.

FIG. 61 depicts an average net LOG reduction for Aspergillus brasiliensis (previously A. niger).

FIG. 62 depicts an average LOG reduction for PhiX trials with control trial data.

FIG. 63 depicts an average Net LOG reduction for PhiX trials with control trial data.

FIG. 64 depicts an average LOG reduction for MS2 trials with control trial data.

FIG. 65 depicts an average Net LOG reduction for MS2 trials with control trial data.

FIG. 66 depicts an average LOG reduction for K. aerogenes trials with control trial data.

FIG. 67 depicts an average Net LOG reduction for K. aerogenes trials with control trial data.

FIG. 68 depicts an average LOG reduction for S. epidermidis trials with control trial data.

FIG. 69 depicts an average Net LOG reduction for S. epidermidis trials with control trial data.

FIG. 70 depicts a CADR; summary of average triplicate results for all organisms challenged against an embodiment of a directional airflow apparatus.

FIG. 71 depicts a CADR and single pass % Efficiency for an embodiment of a directional airflow apparatus against all organisms.

FIG. 72 depicts a CADR graphical calculation example based on real trial data for MS2

### DETAILED DESCRIPTION

In the specification and in the claims, the terms “including” and “comprising” are open-ended terms and should be interpreted to mean “including, but not limited to . . . .” These terms encompass the more restrictive terms “consisting essentially of” and “consisting of.”

It must be noted that as used herein and in the appended claims, the singular forms “a”, “an”, and “the” include plural reference unless the context clearly dictates otherwise. As well, the terms “a” (or “an”), “one or more” and “at least one” can be used interchangeably herein. It is also to be noted that the terms “comprising”, “including”, “characterized by” and “having” can be used interchangeably.

Unless defined otherwise, all technical and scientific terms used herein have the same meanings as commonly understood by one of ordinary skill in the art to which this invention belongs. All publications and patents specifically mentioned herein are incorporated by reference in their entirety for all purposes including describing and disclosing the chemicals, instruments, statistical analyses and methodologies which are reported in the publications which might be used in connection with the invention. All references cited in this specification are to be taken as indicative of the level of skill in the art. Nothing herein is to be construed as an admission that the invention is not entitled to antedate such disclosure by virtue of prior invention.

Currently, the world is dealing with a pandemic due to the spread of COVID-19 and variants thereof. At present, there isn't a drug regime to combat the effects of the virus let alone a particular vaccine that is 100% effective to prevent infection of the virus from person to person. Methods to treat objects that may have been exposed to viruses are somewhat limited in that efficacy of treatment is not fully understood. Constant cleaning of surfaces, face masks, social distancing, avoidance of large groups of people, etc. can only prevent transmission of viruses to a certain extent. Because of this uncertainty, mass transit travel has become limited, visits to hospitals, clinics, and/or emergency rooms have become questionable as to whether a healthy person may be subjected to unwanted viruses, etc. The result has been, and continues to be, a major disruption to businesses, the economy, health and safety considerations, health care, education, etc.

The present embodiments provide a safe, efficient, and rapid approach to decontaminate surfaces and/or the air so that objects can be transferred from site to site without concern whether the surface is contaminated with pathogens, such as protozoa, fungi, molds, bacteria and/or viruses. In one aspect, the object is an individual. Individuals can pass through a portal that provides a stream of hydroxyl ions that will degrade and destroy pathogens, such as protozoa, fungi, molds, bacteria and/or viruses from the surface an object or from the individual. The process is safe and effective in the treatment of bacteria and viruses, such as COVID-19.

It should be understood that throughout this specification, reference to COVID-19 refers to infectious disease caused by the novel coronavirus, SARS-CoV-2, that appeared in late 2019 and also includes variants thereof. COVID-19 is predominantly a respiratory illness that can affect other organs. People with COVID-19 have reported a wide range of symptoms, ranging from mild symptoms to severe illness. Symptoms may appear 2 to 14 days after exposure to the virus. Symptoms may include: fever or chills; cough; shortness of breath; fatigue; muscle and body aches; headache; new loss of taste or smell; sore throat; congestion or runny nose; nausea or vomiting; diarrhea.

COVID-19 variants include, but are not limited to, the Alpha (B.1.1.7), Beta (B.1.351, B.1.351.2, B.1.351.3), Delta (B.1.617.2, AY.1, AY.2, AY.3), Gamma (P.1, P.1.1, P.1.2) and Mu (B.1.621).

In one aspect, the present embodiments provide an apparatus comprising:
a portal comprising a plurality of orifices arranged in an array and positioned on at least one of a first and/or a second opposing side of the portal; and
a hydroxyl ion generator in communication with the portal and the plurality of orifices, wherein hydroxyl ions can be transmitted through the orifices to emit the hydroxyl ions into the area formed by the portal.

The portal can further include a top portion that is connected to the first and second opposing sides. The top portion can further include a plurality of orifices, wherein the hydroxyl ions can be transmitted through the top portion orifices in addition to the first and second side opposing side orifices.

The term “portal” is meant to be an open enclosure that has at least two sides, a first and second opposing side. The sides provide a conduit for the transfer of hydroxyl ions through the enclosure. That is, the hollow sides function as ductwork, a conduit or a tube to deliver the hydroxyl ions to and through the plurality of orifices, such as nozzles. A fan or plurality of fans are connected to or in communication with the sides of the portal to facilitate the emission of the hydroxyl ions through the sides and ultimately through the orifices, such as nozzles, of the sides and/or top portion of the portal.

The portal can simply be two panels opposed to each other with nozzles pointed across from each other from the panels. In one aspect, a top portion is not required.

In another aspect, a top portion is part of the portal and helps to form an “arch” or a rectangular box through which an object can pass. In yet another aspect, the top portion can be in the form of a wedge such that the portal has an apex.

The side panels and/or the top portion of the portal can be manufactured from various materials including plastics, metals such as stainless steel, aluminum, etc. and is not limiting. In one aspect, the side panels and/or top portion are manufactured from sheet aluminum.

The dimensions of the portal can vary depending upon many factors including what type of facility or business the portal is to be located. Exemplary dimensions include side panel widths of from about 1 foot to about 6 feet, lengths of from about 1 foot to about 12 feet, e.g., about 1 foot, and depth of panel side from about 1 inch to about 2 feet, e.g., about 12 inches. The ultimate width of the side panel can be a result of the attachment of multiple side panels to each other.

Similarly, the top portion(s) can also have a width of from about 1 foot to about 6 feet, a length of about 1 foot to 12 feet and a depth of about 1 inch to about 2 feet, e.g., about 5 inches.

Orifices, for example nozzles, can be positioned in an array about the side panels and/or the top portion of the portal. The dimensions of the orifices or nozzles can vary but the orifice or nozzle opening is generally from about ½ inch to about 1½ inches, e.g., about 1 inch for the opening. The plurality of orifice(s) or nozzle(s) can be arranged in an array on the interior of the side panels and/or the top portion of the portal. It should be understood that the orifices can be positioned in linear fashion (e.g., in rows) or can be positioned randomly.

The distance between the plurality of orifices or nozzles can be varied to provide maximum delivery of hydroxyl ions through the orifices or nozzles to the interior section of the portal formed by the side panels and/or top portion. Typically, orifices or nozzles are positioned about 2 to 4 inches apart from each other linearly and from about 6 to about 12 inches from each other in adjacent rows. The number of orifices or nozzles per side panels can be varied depending on the dimensions of the side panels and/or the top portion. Typically, for a side panel of about 3 feet wide and 7 feet in length, there are about 64 orifices or nozzles positioned about the panel and there are about 40 orifices or nozzles for a 3 foot by 3 foot top portion.

The hydroxyl ions used with the apparatus described herein are generated by a hydroxyl ion generator. Hydroxyl ion generators are known in the art and include components such as UV (UVA) bulbs, a titanium oxide platform/support, titanium dioxide, and a water source to provide humidity. One or more fans are generally provided to direct the hydroxyl ion airstream into and through the portal system.

The UV source typically is a UVA bulb. One or more UV bulbs can be used in the hydroxyl ion generator to obtain maximum formation of hydroxyl ions. In some embodiments, the UV source is a black light bulb 950, as shown in FIG. 41.

Alternatively the light emitting source can be a light emitting diode (LED).

The hydroxyl generator creates hydroxyl radicals through a photocatalytic reaction utilizing UVA bulbs and titanium dioxide (TiO₂) coated onto or impregnated into perforated platforms, such as carbon fibers, ceramic panels, aluminum panels and the like. The process utilizes UVA (black light) in the 320 nm to 385 nm region, e.g., 365 nm, wavelength to excite (irradiate) nano sized titanium dioxide particles. Typical hydroxyl generators generate hydroxyls gas having a hydroxyl concentration of about 900 to about 1000 ppm.

The hydroxyl radical, ·OH, is the neutral form of the hydroxide ion (OH−). Hydroxyl radicals are diatomic molecules that are highly reactive and very short-lived with an average half-life of less than two seconds. Hydroxyls work primarily by abstracting hydrogen atoms, thereby dismantling the molecular structure of volatile organic compounds (VOCs). The chain reaction caused by a cascade of organic oxidizing agents is stable enough to react with nearly all organic chemicals, many inorganic chemicals and smoke (airborne particles) throughout the entire treatment space. The hydroxyl generator directs hydroxyl ions from the unit to react with the contaminated air where the hydroxyl ions purify the air and prevent pathogens, such as protozoa, fungi, molds, viruses, microorganisms, and other contaminants from multiplying again. As long as the system is in operation, chain reactions continue, ensuring a constant flow of hydroxyl ions.

The titanium dioxide is coated onto the platform/support. Alternatively, the titanium dioxide is impregnated into the platform.

In order for a hydroxyl generator to produce high levels of hydroxyl radicals, the humidity in the air needs to be about 60%. To avoid having to purchase and locate a separate humidification system, the embodiments described herein have the humidification system built in. In addition to having the humidification unit built into the apparatus, the system is focused on surface sanitation as opposed to air purification. Even though the unit is built to sanitize surfaces, it also has the added benefit of cleaning and purifying the air as well.

The humidification system can be, for example, an ultrasonic humidification system such as those known in the art. An ultrasonic humidifier is one that uses high-frequency sound vibrations to produce an extra fine water mist that is then expelled to add moisture to an area, in this case, in close proximity to the UV lights and the titanium oxide coated/impregnated support. The resulting moisture and hydroxyl ions are then passed through the side panel and/or top portion enclosures described herein. A fan or series of fans are generally positioned above or below the humidification/hydroxyl generator system to force the hydroxyl ions through the side panel conduits/top portion conduit and ultimately through the orifices or nozzles.

The perforated platform/support for the titanium dioxide can be manufactured from a multitude of materials as noted above. The support can be a mesh or screen or any support with a porosity (hole size) sufficient to permit maximum airflow through the portal system/orifices or nozzles. Suitable porous supports include, for example, metallic or porcelain as described above with hole dimensions of from about 0.1 microns to about 5 centimeters. The hole does not need to be symmetrical and can be circular, oblong, trapezoidal, square, etc. Ideally, the perforated platform/support (coated with titanium dioxide) is configured so as to minimally disrupt the airflow of the system described herein.

Alternatively, the support can be flat or rod sheets/rods of metal.

As noted above, one or more fans are utilized to create an airflow through the apparatus and conduct the generated hydroxyl ions through the apparatus and through the multitude of orifices or nozzles. Suitable airflow volumes, cubic feet per minute (cfm), for the fan(s) are from about 600 to about 1000 cfm. In one aspect, there is a balance between the highest CFM possible to distribute the hydroxyl ions while keeping the airflow comfortable for people to pass through the apparatus and keeping the fans noise decibels at a comfortable level. Therefore, the apparatus is designed to provide maximum airflow while keeping the airflow comfortable, and the noise levels as low as possible.

In one aspect, the side panels and/or top portion are divided into two halves so that at least two hydroxyl generators are enclosed per side panel. The number of hydroxyl generator(s)/fan(s)/water source(s) should not be considered limiting as the number of components can vary dependent upon the overall size of the portal system. One, two, three, four, or more hydroxyl generators and the accompanying components can be housed in multiple side panels, for example, or in very large side panels with multiple “bays” that can include a separator portion every few feet, if necessary, to help keep the hydroxyl ion content maximized.

The water source(s) should be constructed as in line continuous water source(s). That is, water should be constantly supplied to a holding tank(s) in the hydroxyl generator/humidification system. The water should be passed through a filter system to remove any impurities that can damage the ultrasonic humidification system. Impurities can affect the generation of the hydroxyl ion and are therefore removed from the water source.

The water source/ultrasonic humidifier can include an automatic shut off valve to the water holding tank so that water does not overflow from the tank. Typical holding tank size is about 1 to about 5 gallons, e.g., 3.6 gallons and, as noted above, multiple tanks can be incorporated into the portal system depending on the width of the side panel(s). In one embodiment, the ultrasonic humidifier is positioned in the container that holds the water and is situated about 5 to about 30 mm below the water line in order to facilitate the humidification. In one embodiment, the humidification device can be attached a float that helps regulate the level of water in the container. The float can simply float within the water container or, alternatively, the float can be held in position by having one or more enclosed holes in the float which can slide up and down in relation to the water level within the water container. In another embodiment, the humidification device is located within the float.

Additionally, the air that is forced through the portal system is filtered as well to remove any particulates, etc. from the air. A high flow electrostatic air filter can be used to cleanse the air prior to entry into the portal system. The filter is typically incorporated into one or more of the side panels of the portal system.

In operation, generally, a fan is positioned above the hydroxyl generator (UV lamps, humidifier, and titanium oxide coated mesh) to pull through the hydroxyl ions into the portal system and orifices or nozzles. Alternatively, the fan can be positioned below the hydroxyl generator to push the hydroxyl ions through the portal system and orifices or nozzles.

In operation, the apparatus can provide a hydroxyl concentration of sufficient concentration to eliminate pathogens, including for example, protozoa, bacteria, fungi, molds, viruses, etc. (e.g., coronaviruses, such as COVID-19) upon exposure to the hydroxyl ions.

A smart tablet can be included with the apparatus which explains how the apparatus works and how to pass/walk an object through the apparatus system.

In another embodiment, an apparatus is provided that includes a directional airflow apparatus and a hydroxyl ion generator in communication with the directional airflow apparatus. The directional airflow apparatus can help support the components of the hydroxyl ion generator system within the conduit/directional airflow apparatus. Hydroxyl ions can be transmitted through the directional airflow apparatus to emit the hydroxyl ions into the surrounding area to kill or reduce the concentration of pathogens, such as protozoa, fungi, molds, viruses and/or bacteria in the air or surfaces in contact with the hydroxyl ion(s). It should be understood that the directional airflow apparatus is used as a housing to contain/support the hydroxyl ion generator system components and is not to be limiting in terms of impeding distribution of hydroxyl ions upon their generation due to the hydroxyl ion reactivity/activity being short lived.

The apparatus can be mounted from the ceiling or can be a standalone unit placed in an enclosed area. Multiple units can be used to treat larger areas.

Typically the surrounding area is an enclosed area such as a restaurant, hospital room, classroom, warehouse, office, etc. That is, the space generally does not have free flowing fresh air and is usually heated/cooled by a heating/ventilation/air conditioning system (HVAC).

The directional airflow apparatus can be ductwork, piping, tubing, and the like that can be configured to distribute the hydroxyl ions into the enclosed area. In one aspect, the directional airflow apparatus can be configured so that the hydroxyl ions are expelled from the hydroxyl generator into a first smaller volume/space that expands into a second larger volume/space of the apparatus, so that mixing of the hydroxyl ions and ambient air are sufficiently mixed as the hydroxyl ions dissipate throughout the enclosed space.

It should be understood that the apparatus expels and distributes hydroxyl ions into the air, killing or reducing the ill effects of protozoa, viruses, molds, fungi, bacteria, etc. and on surfaces in the areas where the hydroxyl ion is active/reactive.

Exposure of the pathogen(s) to hydroxyl ions via the apparatus' and methods described herein can result in eradication of between about 40% to about 100%. For example, a reduction of viable pathogens after exposure to hydroxyl ions for 15 minutes can result in about 44% reduction. 30 minutes in about 80% reduction, 60 minutes in about 90% reduction and after 90 minutes greater than 90% reduction.

### Examples

Objective:

To determine the antibacterial efficacy of ionizing device against S. epidermidis after 15, 30, 60 and 90 minutes of exposure time at room temperature. Staphylococcus epidermidis (S. epidermidis)—ATCC 12228

Test Method:

Bacterial Inoculum Preparation

For S. epidermidis, a pure culture was first plated onto tryptic soy agar supplemented with 5% sheep blood (TSAB) and incubated at 35° C. for 24 hours. A well-isolated colony was then harvested and plated onto fresh TSAB and incubated at 35° C. for 24 hours. Well-isolated colonies were then harvested, suspended in 10% Tryptic Soy Broth (TSB) and vortexed for 1 minute to ensure homogenization. This suspension was used to inoculate the test carriers (sterilized glass slides).

Inoculation of the Test Carriers

Individual sterile glass carriers were inoculated with 50 μL of the microbial suspension and spread evenly across the carrier. This was repeated in triplicate for each time point and the controls. The inoculated carriers were then allowed to air dry (˜45 minutes) inside a biological safety cabinet. Initial testing of the glass carriers showed starting concentrations of 6.60×10⁶ cells/slide for S. epidermidis at time 0.

Simultaneously, the control slides were similarly prepared and inoculated. Inoculated test surfaces were placed one foot away from the ionizing device at right angle and subjected to 15, 30, 60 and 90 minutes of exposure time. Triplicate controls were performed to determine the untreated microbial populations on the test surfaces. After the exposure, the test carriers were placed into centrifuge tubes with sterile buffer water and vortexed to recover any remaining microbes. For S. epidermidis, the recovered samples were serially diluted, plated onto Petrifilm AC plates and incubated for 48 hours at 36±1° C. All test samples and untreated controls were performed in triplicate.

Recovery of Test Organisms: Following exposure, the entire inoculated test carriers and untreated controls were removed using pre-sterilized forceps and placed into 20 mL of phosphate-buffered saline (PBS). The samples were vortexed for 30-60 seconds to recover any remaining bacteria into suspension. The suspension was then serially diluted. For S. epidermidis, 1 mL of each dilution was plated onto AC Petrifilm plates and incubated at 35° C. for 24 hours. After incubation, the recovered colonies were counted. All tests were completed in triplicate.

TABLE 1
Quantitative counts for S. epidermidis exposed to ionizing
devices and untreated control at different time points. The CFU
results are based on the average of three Petrifilm counts.

| | | | Bacterial | | |
|---|---|---|---|---|---|
| | | | Recovery | | |
| | | | CFU/Test | | |
| | | Exposure | Surface | Log | |
| | | Time | (average of | Reduc- | % |
| Time Point | Sample | (min) | 3 surfaces) | tion | Reduction |
| 0 | Untreated | 0 | 6,600,000 | | |
| | control | | | | |
| 30 minutes | Untreated | 0 | 2,000,000 | | |
| | control | | | | |
| 90 minutes | Untreated | 0 | 1,340,000 | | |
| | control | | | | |
| 15 minutes | Treated | 15 | 3,700,000 | 0.25 | 44 |
| | Sample | | | | |
| 30 minutes | Treated | 30 | 1,400,000 | 0.17 | 78 |
| | Sample | | | | |
| 60 minutes | Treated | 60 | 813,000 | 0.40 | 87 |
| | Sample | | | | |
| 90 minutes | Treated | 90 | 513,000 | 0.42 | 92 |
| | Sample | | | | |

CFU: Colony forming Units, Detection limit = 10 CFU
% Reduction—Percent difference between untreated population and treated (exposed) population

The ionizing device was able to kill 87% of S. epidermidis bacteria after 60 minutes of treatment.

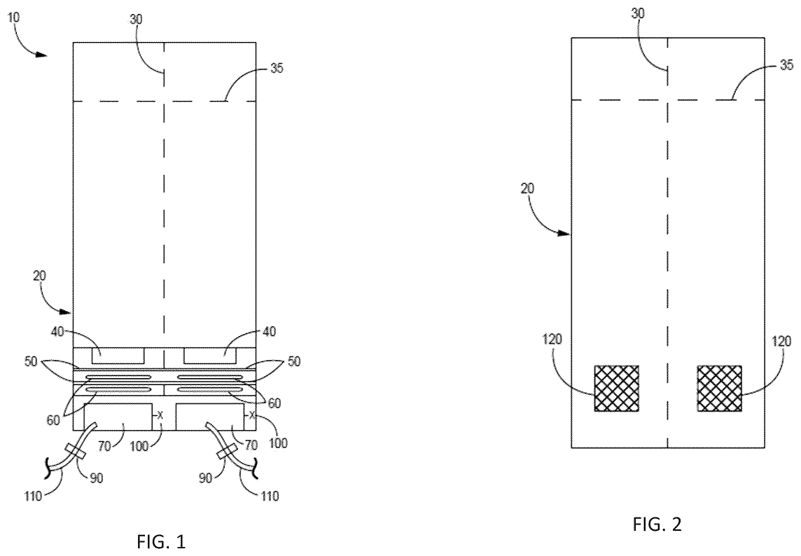

The following FIGS. 1-9 provide a general embodiment of the walk through portal system described herein.

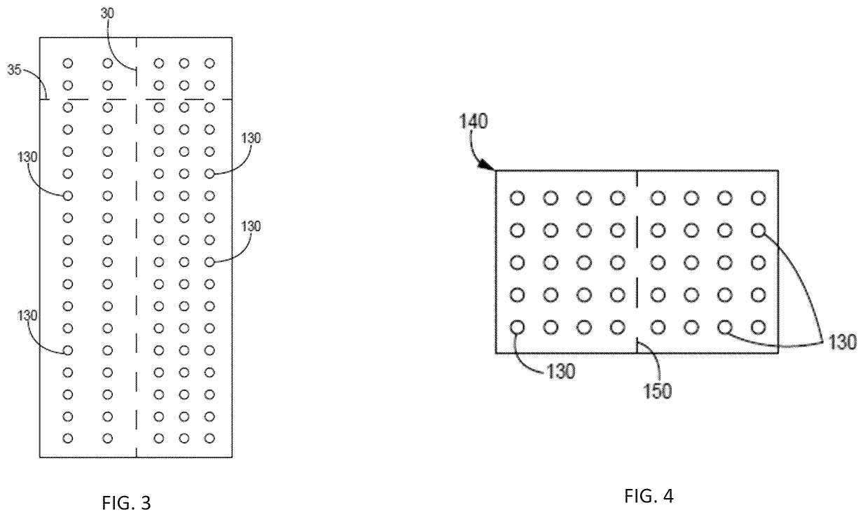

FIG. 1 is a cut away view of a portal apparatus 10 includes side panels 20 which can include an optional divider/separator 30 within the side panel 20. Panel 20 shows the bend for a radius or angle 35 for an optional top portion which can connect two side panels. Within side panel 20 there is contained a fan 40, a mesh support (with titanium dioxide) 50, a UV source (bulb) 60 and a water container 70. A humidification device 80, not shown, resides within water container 70 such that humidification device 80 is position between about 5 to about 3 mm below the water line. Optionally, humidification device 80 resides on or within a float (not shown) that helps regulate the water level in water container 70. Water container 70 is connected to an in line water source 110 which has an in line water filter 90 and also an in line water pressure reducer 100. In operation, the humidifier 80 generates water vapor which passes through/by mesh support 50 and UV source 60 to generate hydroxyl ions. The hydroxyl ions are directed through hollow side panel 20 to orifices 130 shown in FIG. 3 and FIG. 4.

FIG. 2 provides an exterior view of hollow side panel 20 of portal apparatus system 10 with air filter 120.

FIG. 3 is an interior view of hollow side panel 20 of portal apparatus system 10 with openings 130. In some embodiments, the openings are spread in an array. In some embodiments, the openings 130 include orifices formed in the side panel 20. The openings 130 can be openings formed in a mesh or a vent positioned in the side panel 20. In some embodiments, the openings 130 include nozzles.

FIG. 4 depicts an optional top portion 140 of portal apparatus system 10 with openings 130 spread in an array and an optional point of inflection 150 for a maximum radius or point to top portion 140.

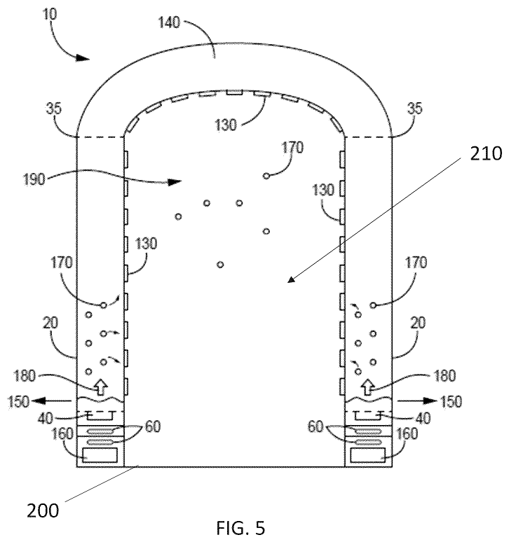

FIG. 5 depicts a side view of portal apparatus system 10 (not to scale) showing side panels 20, the start of radius 35 to optional top portion 140 and orifices 130 (shown as nozzles and not drawn to scale). Cut away section 150 depicts fan 40, mesh support 50, UV source 60 and humidification system 160. Fan 40 creates an air stream 180 to cause hydroxyl ions 170 to flow into the interior 190 of portal apparatus system 10 through openings 130.

The top portion 140 can have an arched configuration. In some embodiments, the top portion 140 and each of the opposing side panels 20 are integrally formed. In an exemplary embodiment, the portal apparatus system 10 is positioned on a surface 200 so that the opposing side panels 20 extend from the surface 200 and the top portion 140 is positioned opposite the surface 200. In some embodiments, the opposing side panels 20 are spaced so that a person can pass between the opposing side panels 20. In some embodiments, the opposing side panels 20 are spaced so that an object can pass between the opposing side panels 20. For example, in one embodiment, the opposing side panels 20 are spaced at least one inch apart. In some embodiments, the top portion 140 and the surface 200 are spaced so that a person can pass between the top portion 140 and the surface 200. In some embodiments, the top portion 140 and the surface 200 are spaced so that an object can pass between the top portion 140 and the surface 200. For example, in one embodiment, the top portion 140 and the surface 200 are spaced at least one inch apart. In one embodiment, an opening 210 formed by the portal apparatus system 10 extends at least one inch in at least one dimension.

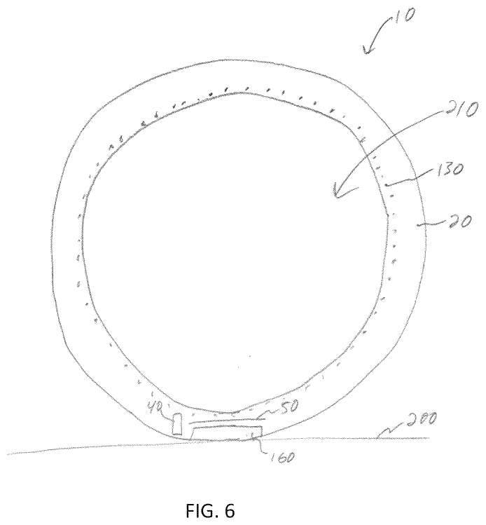

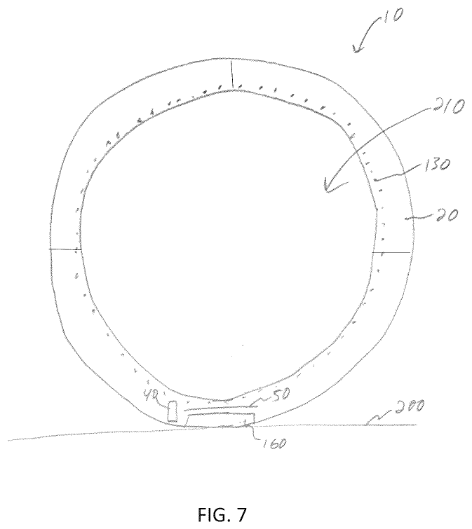

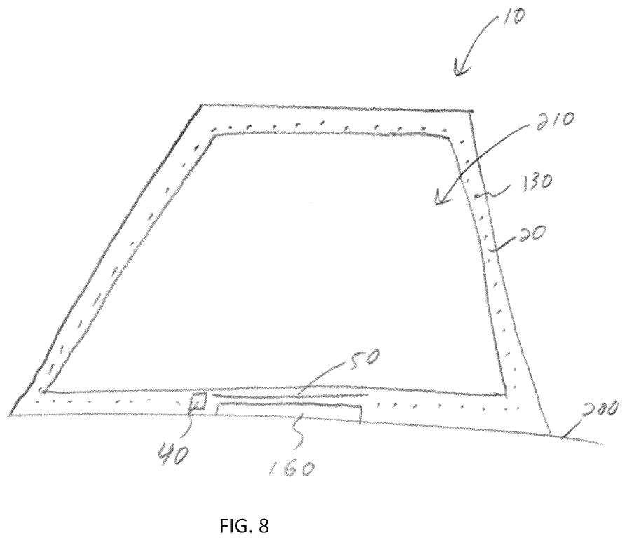

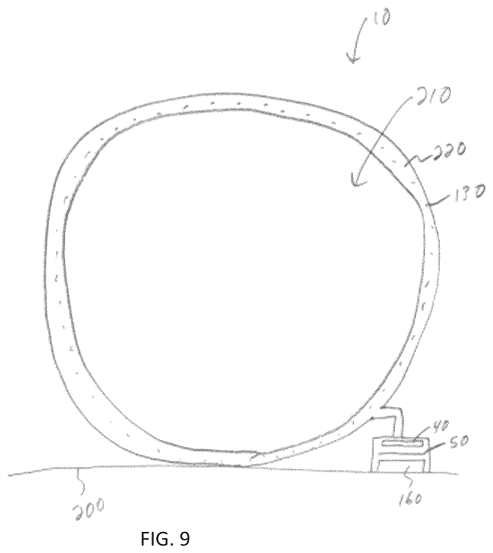

In the embodiment shown in FIG. 5, the portal apparatus system 10 has a substantially rectangular configuration when positioned on the surface 200. In the embodiment shown in FIG. 8, the portal apparatus system 10 has a trapezoidal configuration when positioned on the surface 200. It will be appreciated that the side panel 20 can be shaped in any configuration that forms an opening 210. The openings 130 are angled to direct the hydroxyl ions into the opening 210. For example, in FIG. 6, a single side panel 20 is shaped in a circular configuration that forms the opening 210. As set forth above, the opening 210 has a diameter of at least one inch. It will be appreciated that the opening 210 can be formed by a single side panel 20 or the side panel 20 can be formed from a plurality of side panels 20 coupled together, as illustrated in FIG. 7. Any of the portal shapes described herein can be formed with a single integral side panel 20 or a plurality of panels coupled together. In the embodiment shown in FIG. 9, the panels are replaced by tubing 220. The humidification device 80 is fluidly coupled to the tubing 220.

In some embodiments, the portal apparatus system 10 can include a filter to purify the air prior to passage through the portal apparatus system 10. The filter can be approximately 0.3 microns. If the air isn't clean (contaminants removed), the contaminants can reduce the hydroxyls being put forth from the unit. In some embodiments, the embodiments described herein include timers that indicate when to replace certain parts.

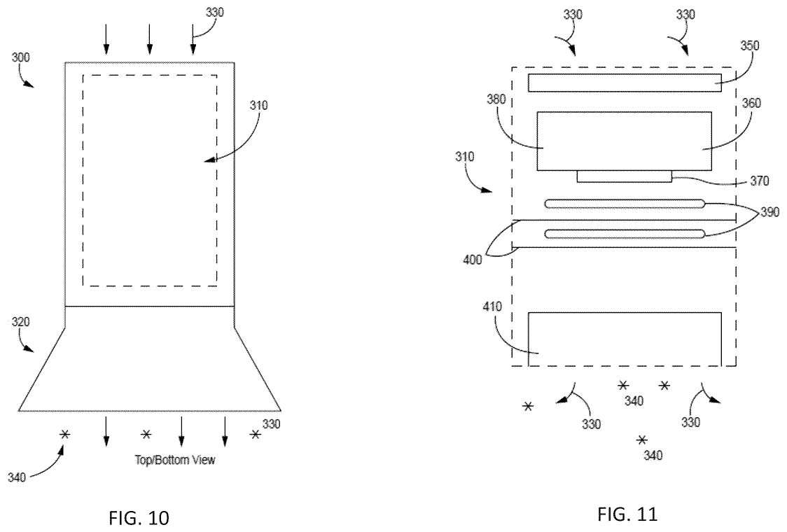

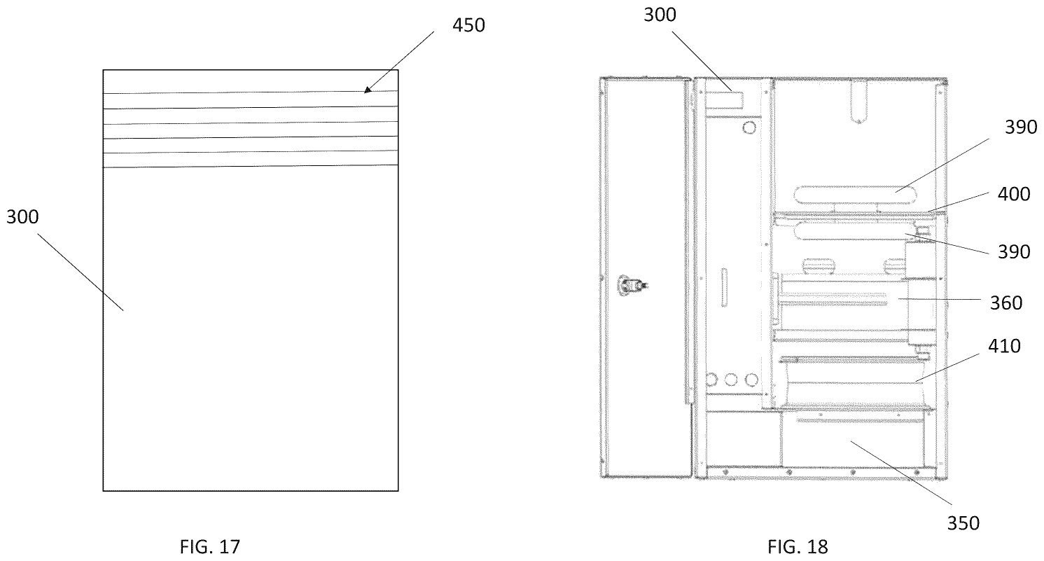

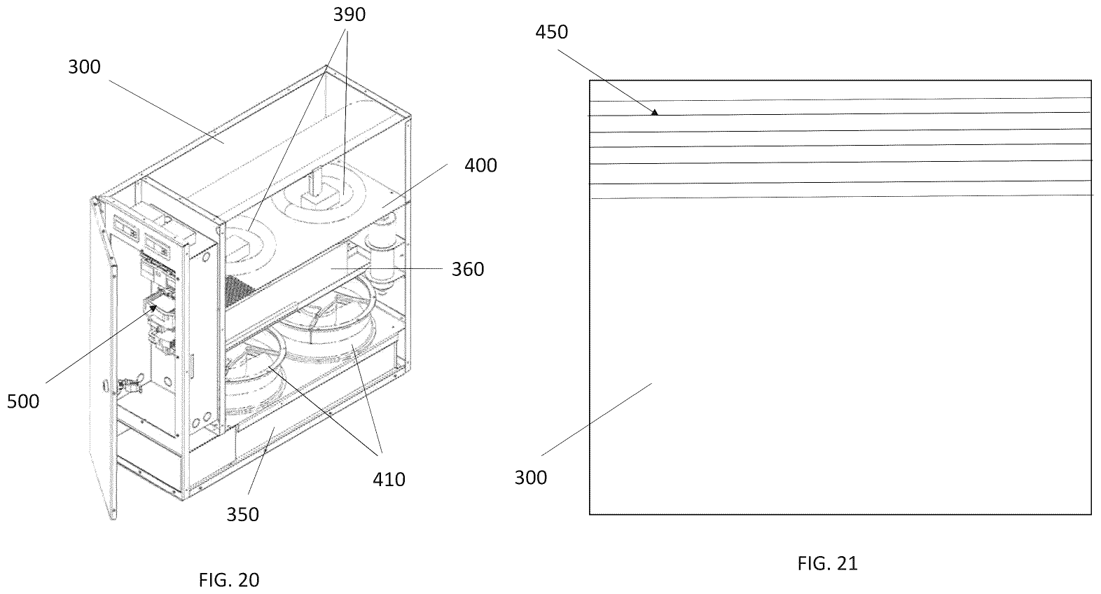

The following FIGS. 10-22 provide a general embodiment of the stand-alone apparatus described herein.

FIG. 10 is a top or bottom view of a stand-alone apparatus suitable for use in an enclosed area. The apparatus can be mounted to a ceiling or can be positioned in a convenient location within the enclosed space. FIG. 10 shows directional airflow apparatus 300 with an optional flange 320 in communication with the directional airflow apparatus 300. 310 is a cut away view of hydroxyl ion generator components for use with the apparatus described in FIG. 11.

FIG. 11 is a top or bottom view of cutaway view 310 of FIG. 10. The hydroxyl ion generator components include with directional airflow apparatus 300, a HEPA (high efficiency particulate air) filter 350. Airflow 330 enters directional airflow apparatus 300 drawn by fan 410 to emit hydroxyl ions 340 from the unit. Airflow 330 passes over water container 360, equipped with water pressure reducer 370 and an ultrasonic device (not shown), optionally with a float (not shown), through UVA bulbs 390 and perforated support with a TiO₂ coating 400 and expelled from directional flow apparatus 300 through optional flange 320 to emit activated hydroxyl ions 340 into an enclosed area.

In one aspect, fan 420 can be positioned in front of HEPA filter 350 to push air through directional airflow apparatus instead of drawing air through the apparatus. In another aspect, the apparatus can be integrated with an HVAC system to purify the incoming air within the enclosed space.

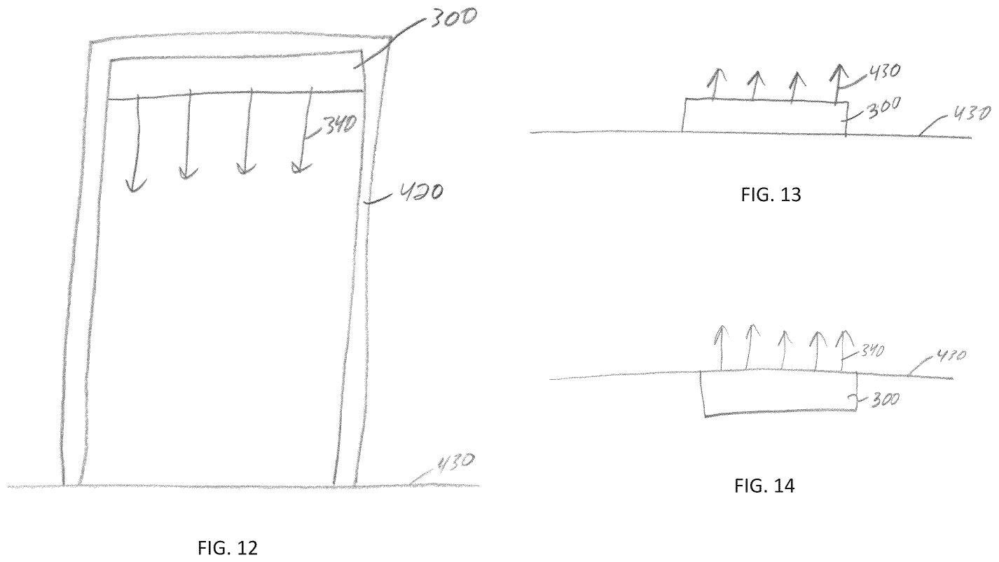

FIG. 12 illustrates a doorway 420. In one embodiment, the directional airflow apparatus 300 is configured to emit the activated hydroxyl ions 340 into the doorway 420. Accordingly, a person or object passing through the doorway 420 is subjected to the activated hydroxyl ions 340. In another embodiment, the directional airflow apparatus 300 can be positioned on a floor 430, as illustrated in FIG. 13, or embedded within the floor 430, as illustrated in FIG. 14. In such an embodiment, the directional airflow apparatus 300 directs the activated hydroxyl ions 340 upward. Accordingly, any person or object passing over the directional airflow apparatus 300 is subjected to the activated hydroxyl ions 340.

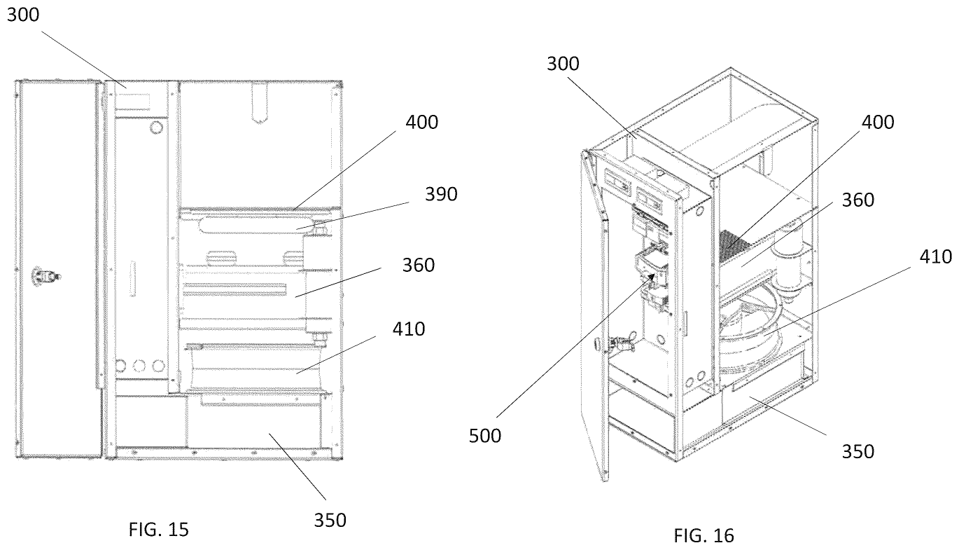

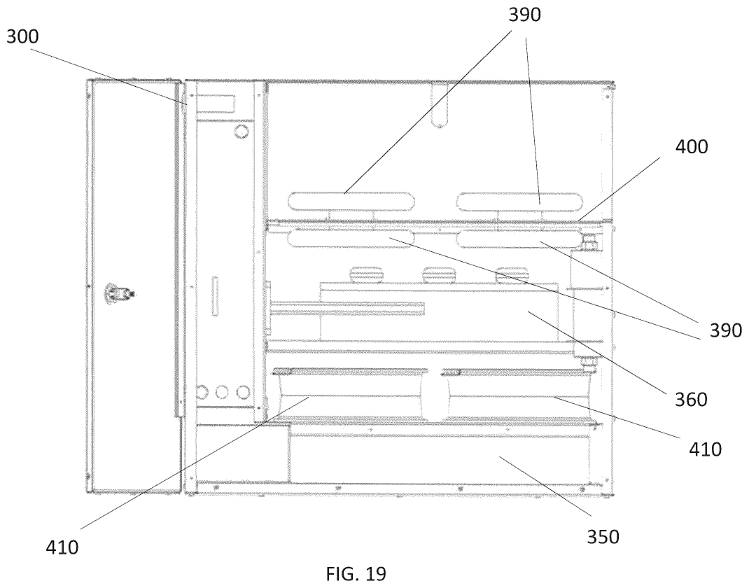

FIGS. 15-18 illustrate another embodiment of a directional airflow apparatus 300 that can be mounted to a wall or positioned on or within a floor of an enclosed space. The apparatus 300 can also be mounted or embedded in a ceiling or wall. The water container 360 is positioned at a bottom of the directional airflow apparatus 300 and includes an atomizer to produced water droplets. The fan 410 generates airflow 330 that directs the water droplets through UVA bulbs 390 and perforated support with a TiO₂ coating 400 to produce hydroxyl ions 340 that are emitted through a vent 450 at the top of the directional airflow apparatus 300. The embodiment illustrated in FIGS. 15-17 includes a single fan 410 and a single UVA bulb 390 positioned under the perforated support with a TiO₂ coating 400. As seen in FIG. 18, the directional airflow apparatus 300 can include a UVA bulb 390 both under and above the perforated support with a TiO₂ coating 400. It will be appreciated that the directional airflow apparatus 300 can be modified for larger enclosed spaces. For example, the embodiment shown in FIGS. 19-21 includes additional fans 410 and UVA bulbs 390 to facilitate increasing the hydroxyl ion output. In some embodiments, the directional airflow apparatuses 300 described herein include timers that indicate when to replace certain parts.

In one embodiment, an apparatus 300 can be configured to operate in a space of approximately 0-500 square feet. Such an apparatus 300 can be configured to move and clean approximately 5,000 cubic feet of air every 15 minutes. Such an apparatus 300 can be configured with one UVA bulb 390 and one 550 cubic foot per minute fan 410. Another apparatus 300 can be configured to operate in a space of approximately 500-900 square feet. Such an apparatus 300 can be configured to move and clean approximately 5,000-9,000 cubic feet of air every 15 minutes. Such an apparatus 300 can be configured with two UVA bulbs 390 and one 700 cubic foot per minute fan 410. A further apparatus 300 can be configured to operate in a space of approximately 900-1,500 square feet. Such an apparatus 300 can be configured to move and clean approximately 9,000-15,000 cubic feet of air every 15 minutes. Such an apparatus 300 can be configured with two UVA bulbs 390 and one 1,000 cubic foot per minute fan 410. Yet another apparatus 300 can be configured to operate in a space of approximately 1,500-2,000 square feet. Such an apparatus 300 can be configured to move and clean approximately 15,000-20,000 cubic feet of air every 15 minutes. Such an apparatus 300 can be configured with four UVA bulbs 390 and two 700 cubic foot per minute fans 410. An additional apparatus 300 can be configured to operate in a space of approximately 2,000-2,500 square feet. Such an apparatus 300 can be configured to move and clean approximately 20,000-25,000 cubic feet of air every 15 minutes. Such an apparatus 300 can be configured with four UVA bulbs 390 and two 1,000 cubic foot per minute fans 410.

In some embodiments, the directional airflow apparatus 300 can include the filter 350 to purify the air prior to passage through the directional airflow apparatus 300. The filter 350 can be approximately 0.3 microns. If the air isn't clean (contaminants removed), the contaminants can reduce the hydroxyls being put forth from the apparatus 300.

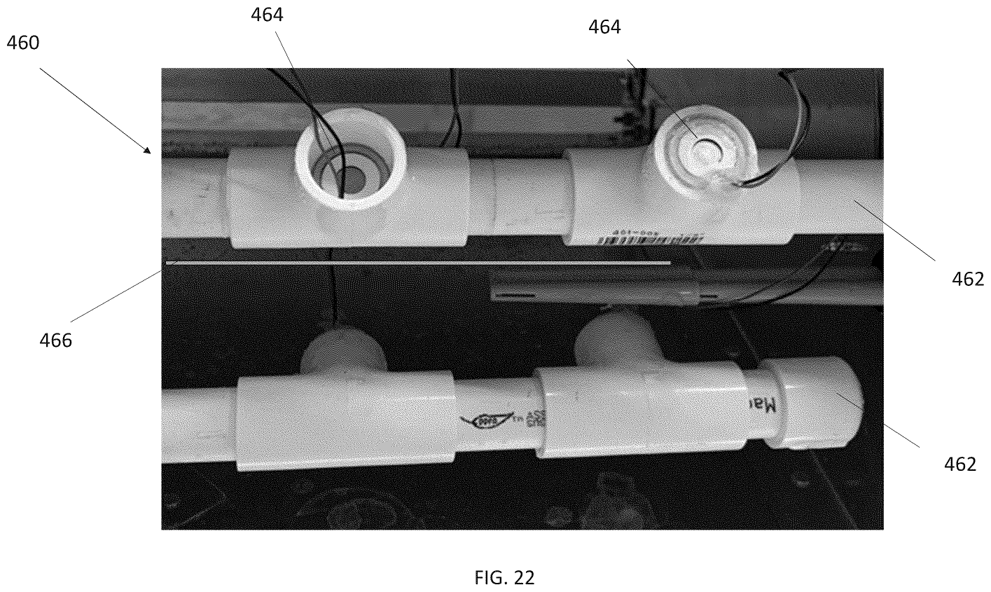

Referring to FIG. 22, a humidification system 460 of the apparatus 300 can include a water channel 462. At least one atomizer 464 is positioned in the water channel 462. The at least one atomizer 464 is configured to atomize water in the water channel 462. That is, the water in the water channel 462 comes in contact with the at least one atomizer 464 so that the at least one atomizer atomizes the water to form water droplets. In an exemplary embodiment, the at least one atomizer 464 also seals the water channel 462 to prevent water from leaking from the water channel 462. In one embodiment, the water droplets pass over at least one light emitting diode 466. In the exemplary embodiment, a strip of light emitting diodes 466 is provided.

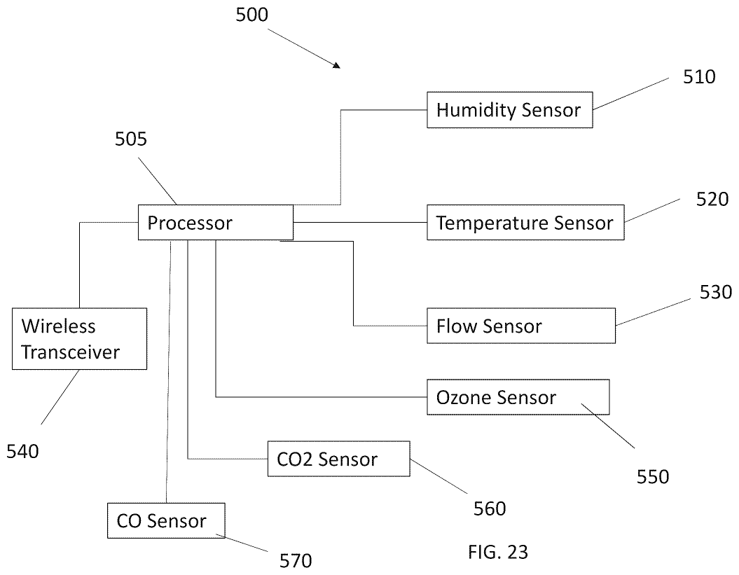

Any of the embodiments described herein can include a control system 500. A schematic of the control system 500 is illustrated in FIG. 23 and will be described with respect to the apparatus 300. The control system 500 includes a processor 505. A humidity sensor 510 monitors a percent humidity generated by the apparatus 300 to ensure that the percent humidity is within a range that provides a predetermined concentration of hydroxyl ions 340. In some embodiments, another humidity sensor can measure a humidity of the airflow into the directional airflow apparatus 300. That is, the humidity sensor 510 can include an inlet humidity sensor 510 and an outlet humidity sensor 510. A temperature sensor 520 monitors a temperature of the hydroxyl ions 340. In some embodiments, another temperature sensor can measure a temperature of the airflow into the directional airflow apparatus 300. That is, the temperature sensor 520 can include an inlet temperature sensor 520 and an outlet temperature sensor 520. A flow sensor 530 monitors a flow rate of the hydroxyl ions 340. In some embodiments, another flow sensor can measure a flow rate of the airflow into the directional airflow apparatus 300. That is, the flow sensor 530 can include an inlet flow sensor 530 and an outlet flow sensor 530.

The sensors 510, 520, 530 provide feedback to the processor 505 so that the control system 500 can alter the function (e.g. activate and deactivate) of the atomizer or the hydroxyl ion generator. At least one of the humidity sensor 510, the temperature sensor 520, and the flow sensor 530 can be located at the directional airflow apparatus 300. In other embodiments, at least one of the humidity sensor 510, the temperature sensor 520, and the flow sensor 530 can be located remotely from the apparatus 300. At least one of the humidity sensor 510, the temperature sensor 520, and the flow sensor 530 can be hardwired or wirelessly coupled to the processor 505.

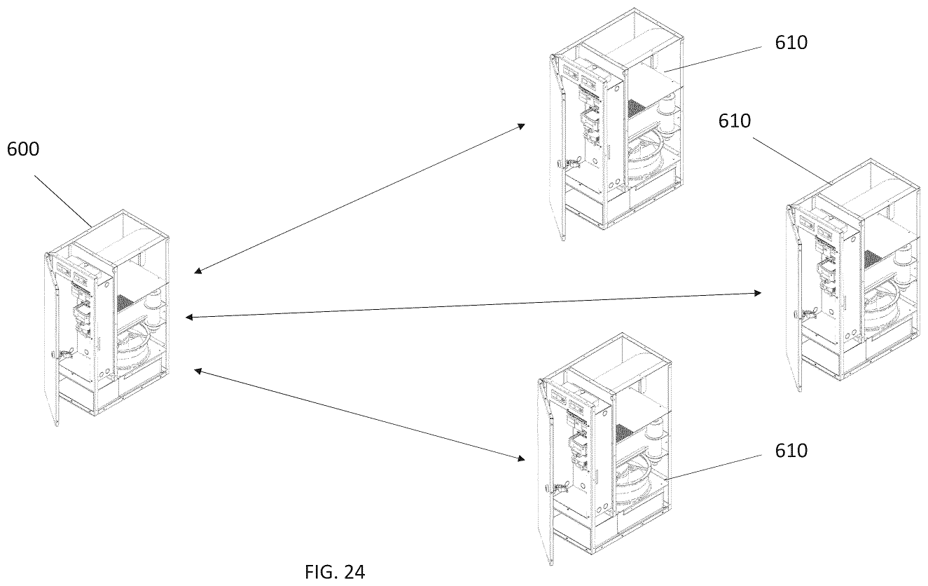

The control system 500 also includes a wireless transceiver 540 that enables the apparatus 300 to wirelessly communicate with other apparatuses 300, as described in more detail below with regard to FIG. 24. In one embodiment, the wireless transceiver 540 is a Bluetooth® device. The wireless transceiver 540 can also enable communication with a remote device, for example, a mobile phone or tablet. Accordingly, the apparatus 300 can be controlled remotely from the remote device. For example, a speed of the fan 410 can be adjusted using the remote device or the fan 410 may be set in a silent mode that reduces an amount of sound produced by the apparatus 300.

In one embodiment, the control system 500 can adjust the flow rate of the hydroxyl ions 340 based on at least one of the humidity as measured by the humidity sensor 510, the temperature as measured by the temperature sensor 520, and the flow rate as measured by the flow sensor 530. In another embodiment, the control system 500 can adjust the flow rate of the hydroxyl ions 340 based on at least two of the humidity as measured by the humidity sensor 510, the temperature as measured by the temperature sensor 520, and the flow rate as measured by the flow sensor 530. In yet another embodiment, the control system 500 can adjust the flow rate of the hydroxyl ions 340 based on all three of the humidity as measured by the humidity sensor 510, the temperature as measured by the temperature sensor 520, and the flow rate as measured by the flow sensor 530. In some embodiments, the flow rate of the hydroxyl ions 340 is adjusted by controlling the atomizer (for example, turning the atomizer on and off).

In one embodiment, the control system 500 can adjust the hydroxyl ion generator based on at least one of the humidity as measured by the humidity sensor 510, the temperature as measured by the temperature sensor 520, and the flow rate as measured by the flow sensor 530. In another embodiment, the control system 500 can adjust the hydroxyl ion generator based on at least two of the humidity as measured by the humidity sensor 510, the temperature as measured by the temperature sensor 520, and the flow rate as measured by the flow sensor 530. In yet another embodiment, the control system 500 can adjust the hydroxyl ion generator based on all three of the humidity as measured by the humidity sensor 510, the temperature as measured by the temperature sensor 520, and the flow rate as measured by the flow sensor 530. In some embodiments, the UVA bulbs 390 of the hydroxyl ion generator are controlled (for example, turned on or off) by the control system 500 to control the hydroxyl ion generator.

In some embodiments, the humidity is maintained at approximately 48% humidity in the enclosed space. In some embodiments, the humidity is maintained within a range of approximately 40% to 60%. In some embodiments, the humidity is maintained within a range of approximately 45%-55%. In some embodiments, the humidity is maintained within a range of approximately 57%-59%. In some embodiments, the humidity is maintained below 60%.

The control system 500 also includes an ozone sensor 550 to measure an amount of ozone if produced by the apparatus 300 or by another apparatus not associated with apparatus 300. The control system 500 can shut off the apparatus 300 if a predetermined level of ozone is detected by the ozone sensor 550. It will be appreciated that in an exemplary embodiment, the hydroxyl ions are produced without producing ozone. The control system 500 can also include a carbon dioxide sensor 560 to measure an amount of carbon dioxide produced by the apparatus 300. The control system 500 can shut off the apparatus 300 if a predetermined level of carbon dioxide is detected by the carbon dioxide sensor 560. The control system 500 can further include a carbon monoxide sensor 570 to measure an amount of carbon monoxide produced by the apparatus 300. The control system 500 can shut off the apparatus 300 if a predetermined level of carbon monoxide is detected by the carbon monoxide sensor 570.

One advantage of the apparatus', systems and methods described herein is that ozone is not generated.

Another advantage of the apparatus', systems and methods described herein is that the hydroxyl ions, once emitted from the apparatus/system will interact with undesired contaminants in the air (e.g., allergens, pollen, protozoa, fungi, bacteria, viruses, etc.) and cause them to decompose and/or degrade so as to not infect an individual or reduce the discomfort that the individual suffers from allergies. Thus the apparatus' and systems can be used to treat such contaminants, reducing and/or eliminating such contaminants.

Still another advantage of the apparatus', systems and methods described herein provides a clean smelling environment that is akin to fresh mountain air.

In some embodiments, the apparatus 300 can be used in a refrigeration system. For example, the apparatus 300 can be used in a household refrigerator, a commercial refrigerator, or a refrigerated transportation vehicle. In such an embodiment, the apparatus 300 can be used to prolong the freshness of food.

FIG. 24 illustrates a plurality of directional airflow apparatuses 300 in an enclosed space. The plurality of directional airflow apparatuses 300 includes a master unit 600 and at least one slave unit 610. In an exemplary embodiment, the master unit 600 controls the at least one slave unit 610. In some embodiments, each of the slave units 610 includes at least one of the sensors 510, 520, 530 described above. The master unit 600 can also include at least one of the sensors 510, 520, 530 described above. The processor 505 can be housed at the master unit 600. Signals from each of the sensors 510, 520, 530 are transmitted to the master unit 600 so that the master unit 600 can individually control each slave unit 610. That is, the master unit 600 can individually control a fan 410 or hydroxyl ion generator of each slave unit 610. In some embodiments, the master unit 600 can individually turn the slave units on and off.

In some embodiments, the plurality of directional airflow apparatuses 300 can be incorporated into an HVAC system of the enclosed spaced. In such an embodiment, the control system 500 can work in conjunction with the HVAC system to control each of the plurality of directional airflow apparatuses 300. It will be appreciated that a single directional airflow apparatus can be incorporated into the HVAC system.

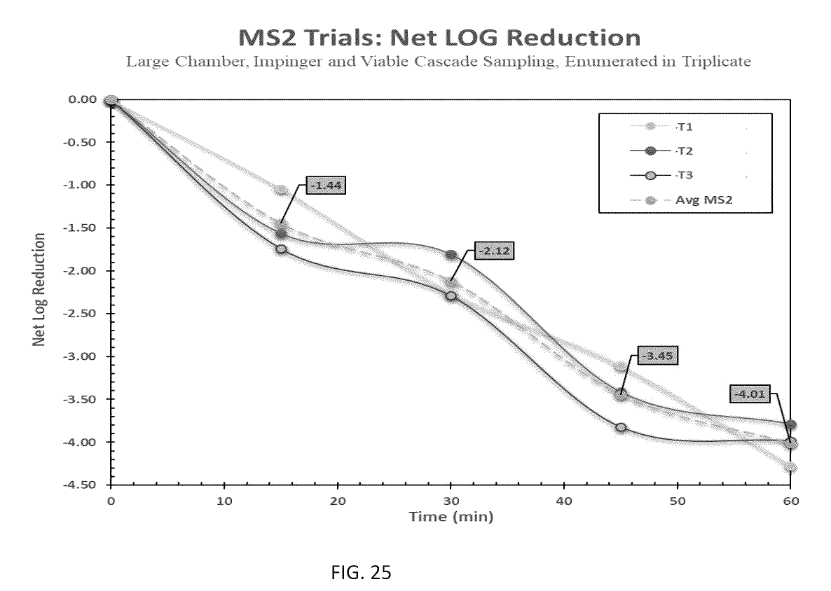

Referring to FIG. 25 a graph is provided to illustrates the data from three separate trials conducted on the apparatus described herein in a 16 m³ chamber over the course of 60 minutes. For the three separate trials, species selection was based on Biological Safety Level 1 (BSL1) surrogates for BSL2-BSL3 pathogenic organisms. MS2 bacteriophage (ATCC 15597-B1) is an un-enveloped, tail-less virus, that is 25 nm in size. It contains linear ssRNA as its genome and has historically been used as an Influenza surrogate. MS2 has similar aerosol properties to other viruses due to their small size and shape. Measuring the capture efficiency of the Poppy device with MS2 helps create a reliable in-vitro test model. It also may help as an in vitro indicator for how pathogenic viruses could be handled in real world settings.

A 24-Jet Collison nebulizer was used to generate bioaerosol within the chamber. The nebulizer fed bioaerosol into the chamber through a tri-clover stainless pass-through port centrally located on the side-chamber wall. The system was constructed using stainless sanitary tubing and connected to a common stainless manifold with a controllable dilution air valve. In order to sample the air within the chamber, four AGI-30 impingers (Ace Glass Inc. Vineland NJ) were used for bioaerosol collection to determine challenge concentrations. These impingers were connected to the test chamber via ⅜″ stainless tube sample ports. These ports were fastened to the chamber using ⅜″ stainless bulkhead fittings. Impingers were filled with 20 mL of sterilized phosphate buffer solution (PBS) containing 0.005% v/v of Tween 80 for bioaerosol collection. These impingers were specifically designed to collect chamber air at a constant flow rate. This allows for precise back calculations of chamber concentration based on the impinger sampling duration. The nebulization air flow rate of the Collison 24-jet nebulizer and dilution air flow rate was controlled and monitored to ensure that the same rates for all of the challenge trials were properly maintained. The Collison 24-jet nebulizer was operated at a head pressure of 35 psi with an approximate output rate of 15 L/min with an additional 85 L/min of dilution air, totaling 100 L/min for all of the trials conducted. All exposures were conducted at room temperature with the internal ceramic unit inside the test chamber set to 20° C. in order to maintain a steady exposure temperature for the duration of the trials. These trials also had humidity regulation to maintain the chamber at approximately 60% relative humidity. All four mixing fans were turned on for the duration of each trial in order to ensure adequate bioaerosol mixing.

Following each chamber characterization trial, the impinger samples were pooled for an overall average of the entire chamber concentration. Using calibrated pippettes, impinger samples were serially diluted and subsequently plated in triplicate for each dilution. These dilutions were plated using a standard small drop assay in triplicate on labeled sterile Tryptic Soy Agar Dishes (Hardy Diagnostics, Santa Maria, CA). Impinger samples were then incubated for approximately 24 hours at 37° C.

The results, shown in FIG. 25, represent the total Net Log reduction of MS2 overtime within the testing chamber. The net log reduction considers the control losses determined by the control tests, or tests without the test device operational, and subtracts that result from the log reduction observed during the trials with the device operational. Doing this allows for a clear representation of the device's efficacy without the contribution of natural die off of the challenge organism. Based on the triplicate trials performed to assess the apparatuses ability to remove viable virus from a given area, we've demonstrated an average reduction of 4.01 in 60 min of device operation. This would equate to a 99.99% percent removal of MS2 from the air. These results suggest that the apparatus would effectively remove 99.99% of similar viruses from the air within 60 minutes of operation in a similarly sized space.

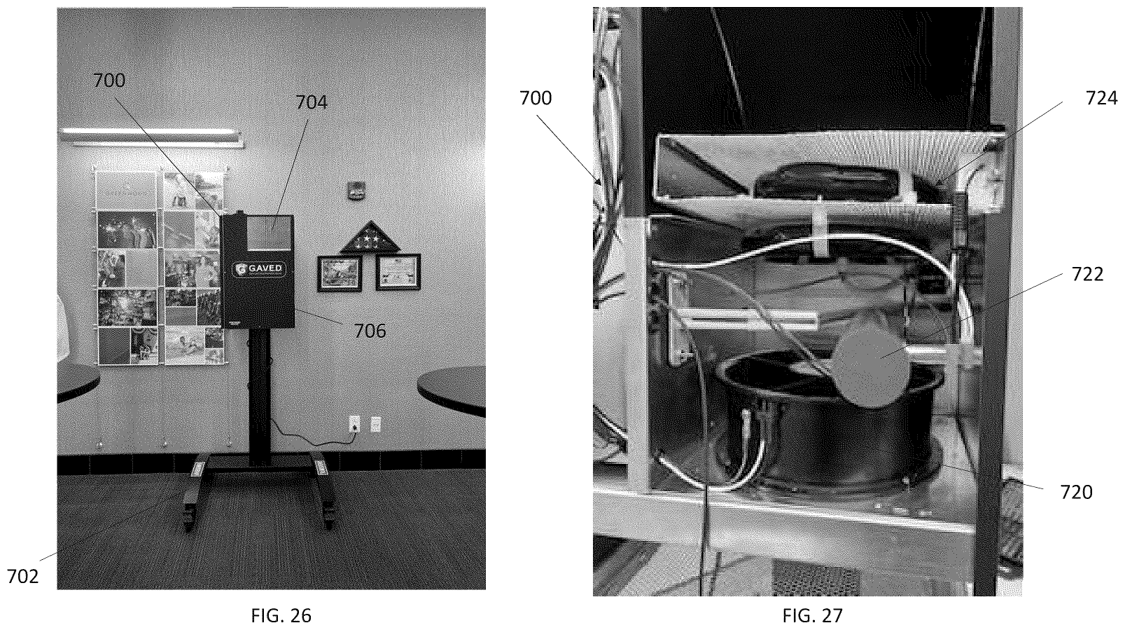

Referring now to FIG. 26 a portable directional airflow apparatus 700 is positioned on a portable cart 702. It will be appreciated that, in some embodiments, the portable directional airflow apparatus 700 is positioned on any moveable device. In other embodiments, the portable directional airflow apparatus 700 is not positioned on a portable device, but rather, is configured to be carried. For example, in some embodiments, the portable directional airflow apparatus 700 includes handles or straps for lifting the portable directional airflow apparatus 700.

The apparatus 700 includes a vent 704 for discharging hydroxyl ions, as described herein. In the illustrated embodiment, the vent 704 is positioned in a front side 706 of the apparatus 700. In other embodiments, the vent 704 is positioned in any side of the apparatus 700 that facilitates discharging hydroxyl ions.

As seen in FIG. 27, the apparatus 700 includes a fan 720 that directs air through the apparatus 700 and out of the vent 704. A water channel 722 is positioned over the fan 720 and configured to atomize water. The atomized water is directed by the fan 720 to a hydroxyl ion generator 724 that produces hydroxyl ions, as described herein. The hydroxyl ions are discharged from the apparatus 700 through the vent 704.

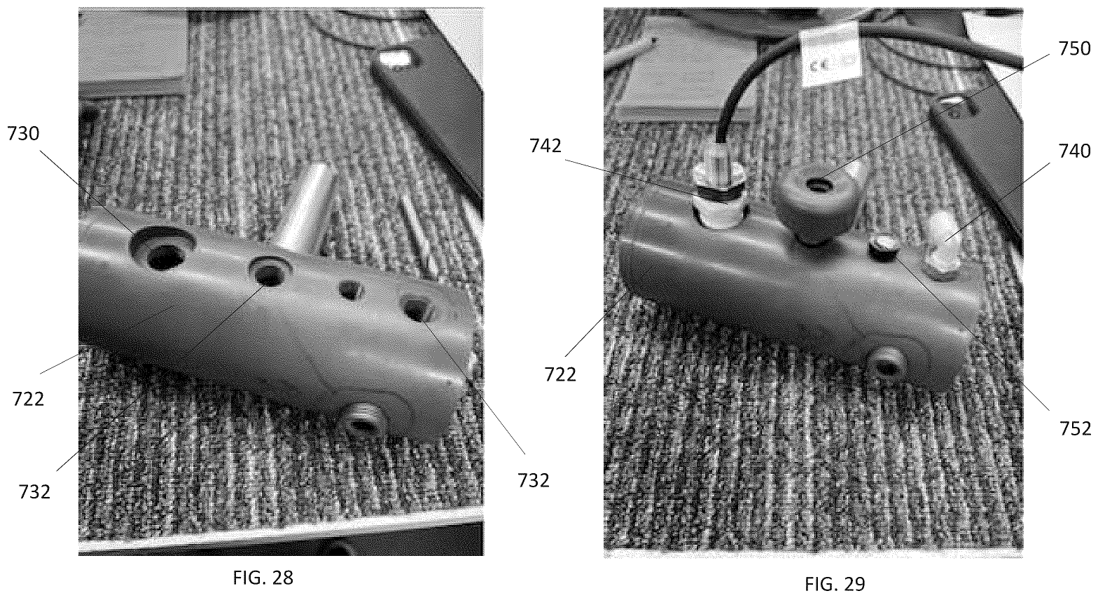

Referring now to FIG. 28 the water channel 722 includes an inner cavity 730 that fills with water. A plurality of openings 732 are formed in a side of the water channel 722 to access the cavity 730. As seen in FIG. 29, a plurality of components are positioned in the openings 732. A water line 740 directs a flow of water into the cavity 730. A solenoid valve 742 is operable between an open state and a closed state. In the open state, the solenoid valve 742 allows the flow of water from the water line 740 into the cavity 730. In the close state, the solenoid valve 742 restricts the flow of water from the water line 740 into the cavity 730. An atomizer 750 atomizes the water in the cavity 730 of the water channel 722. A vent 752 vents the water channel 722 to ease the flow of water into the cavity 730.

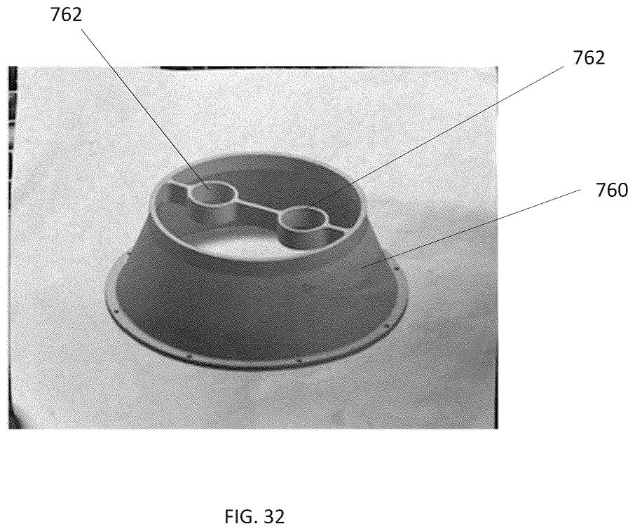

It will be appreciated that the water channel 722 is operable with any of the apparatuses described herein. In some embodiments, the water channel 722 is replaced with the cover 760 shown in FIG. 32. The cover 760 is positioned over the fan 704. The cover 760 includes openings 762 that are configured to retain the solenoid valve 742 and the atomizer 750.

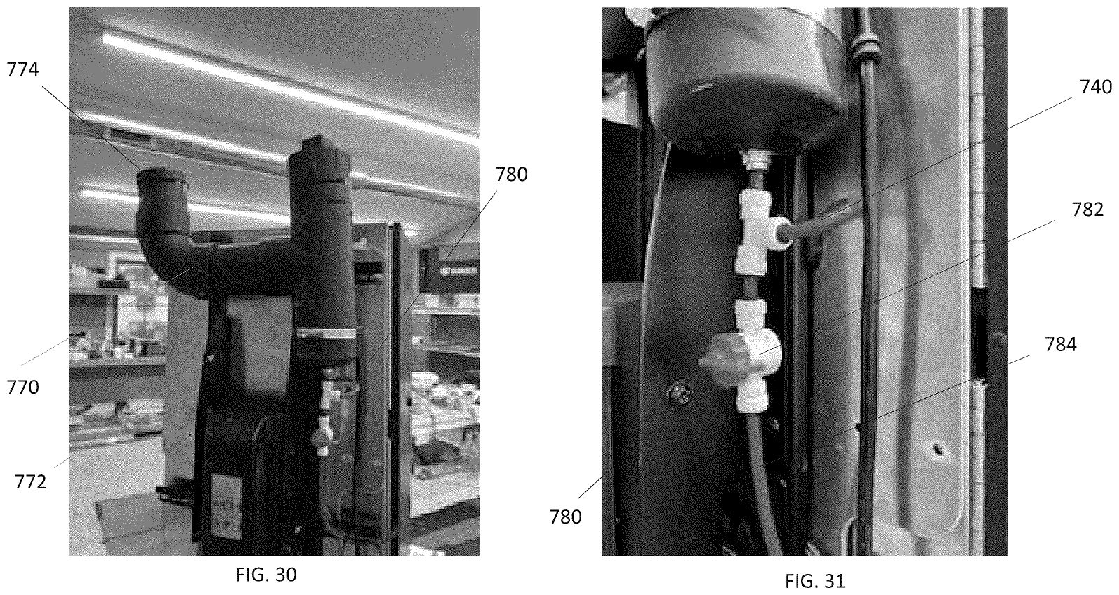

FIG. 30 illustrates a water tank or reservoir 770 that is positioned on a back side 772 of the directional airflow apparatus 700. It will be appreciated that, in some embodiments, the water tank 770 is positioned in any other suitable location on or in the apparatus 700. In some embodiments, the water tank 770 is positioned on the cart 702. In some embodiments, the water tank 770 holds approximately 2 gallons of water. In other embodiments, the water tank 770 holds any suitable amount of water.

The water tank 770 includes a vent 774 that permits the flow of water from the tank 770 into a valve system 780, as shown in more detail in FIG. 31. The valve system 780 directs the water to the water line 740 (described above). A valve 782 is operable to open a drain 784 to drain the tank 770. It will be appreciated that is some embodiments, the portable apparatus 700 is coupleable to an existing water line that directs water to the water line 740 without the need for the tank 770. For example, in some embodiments, the water line 740 is coupleable to a hose or faucet.

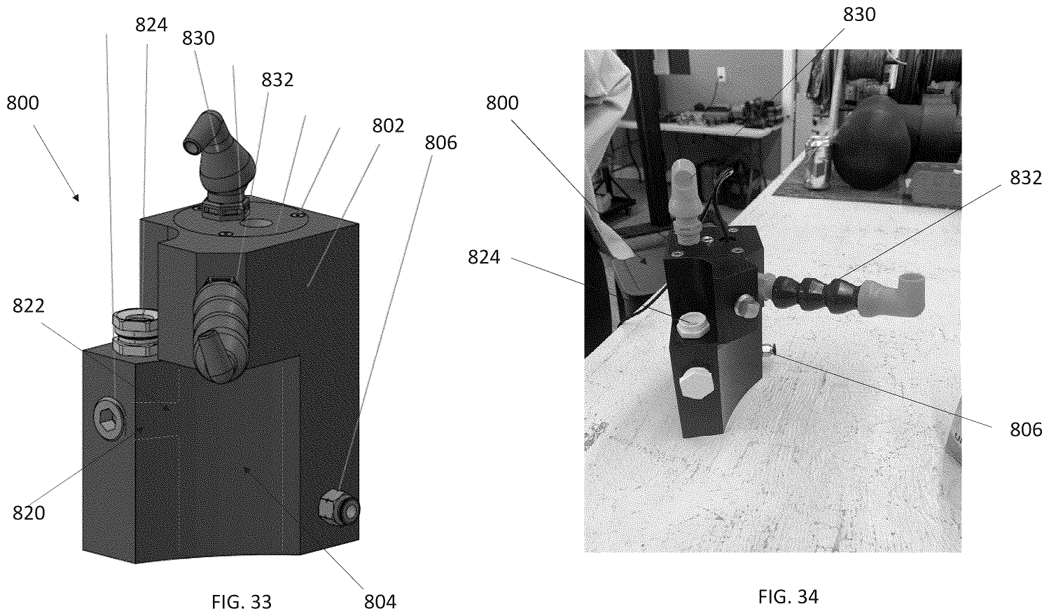

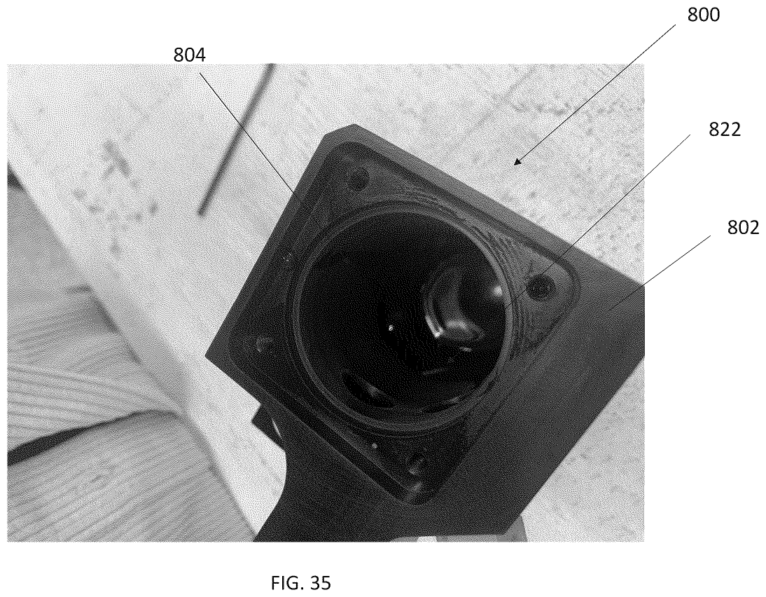

Referring now to FIGS. 33-37, an atomizer system 800 is configured to generate water droplets that are directed into a hydroxyl ion generator 850, for example, one of the hydroxyl ion generators described above. The atomizer system 800 includes a housing 802 that defines a cavity 804 (shown in FIG. 35). A water inlet 806 is coupled to a water source (as described above). The water inlet 806 dispenses water into the cavity 804. An atomizer 808 (shown in FIG. 37) is positioned in the cavity 804 and configured to be submerged by the water in the cavity 804. The atomizer 808 uses ultrasonic electronics to convert the water molecules in the cavity 804 into atomized water droplets.

A channel 820 extends from the cavity 804 through an opening 822. In some embodiments, the channel 820 extends approximately 90 degrees from the cavity 804. Water flows from the cavity 804 into the channel 820. A sensor 824 extends through the housing 802 into the channel 820. The sensor 824 is configured to detect water in the channel 820. A flow of water into the cavity 804 is controlled by a control system (as described above) based on a water level in the channel 820 detected by the sensor 824.

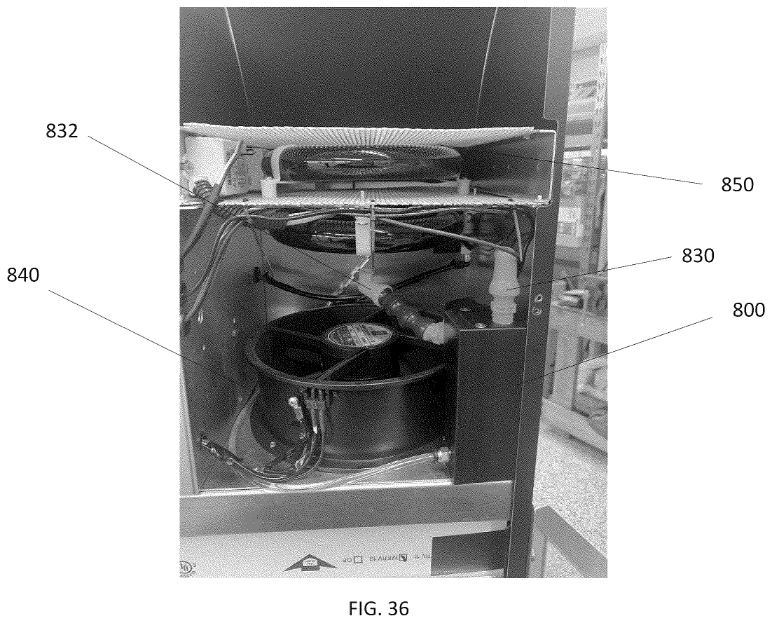

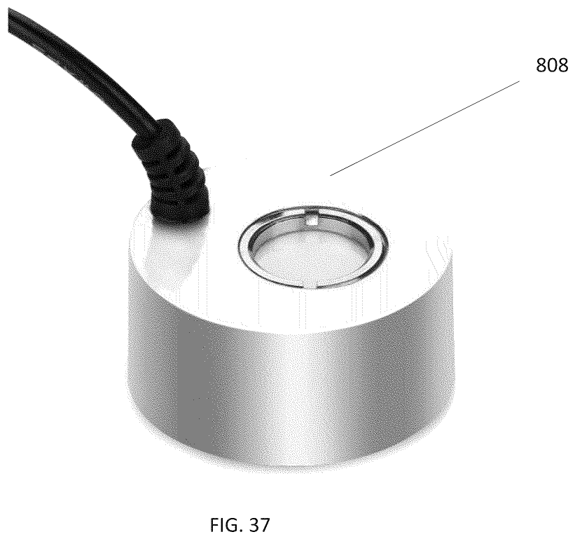

An air intake 830 directs air into the cavity 804. An air outlet 832 directs air containing the water droplets out of the cavity 804. The atomizer system 800 is positioned near a fan 840 (as shown in FIG. 36). In some embodiments, the air outlet 832 is positioned adjacent the fan 840. The fan 840 directs the water droplets from the atomizer system 800 into the hydroxyl ion generator.

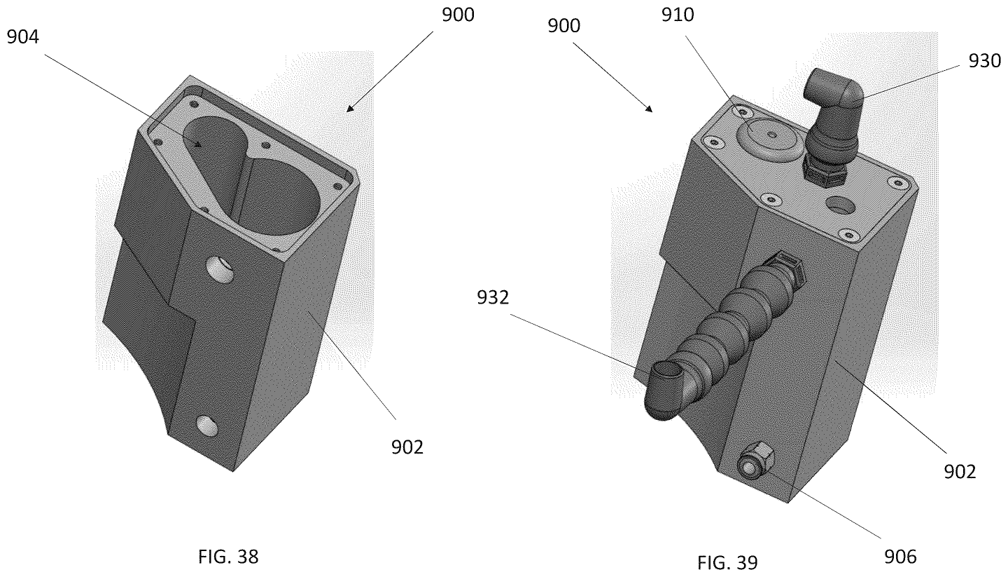

Referring now to FIGS. 38-40, an alternative embodiment of an atomizer system 900 is illustrated. The atomizer system 900 is configured to generate water droplets that are directed into a hydroxyl ion generator, for example, one of the hydroxyl ion generators described above. The atomizer system 900 includes a housing 902 that defines a cavity 904 (shown in FIG. 38). A water inlet 906 is coupled to a water source (as described above). The water inlet 906 dispenses water into the cavity 904. An atomizer, for example the atomizer 808 shown in FIG. 37, is positioned in the cavity 904 and configured to be submerged by the water in the cavity 904. The atomizer 808 uses ultrasonic electronics to convert the water molecules in the cavity 904 into atomized water droplets.

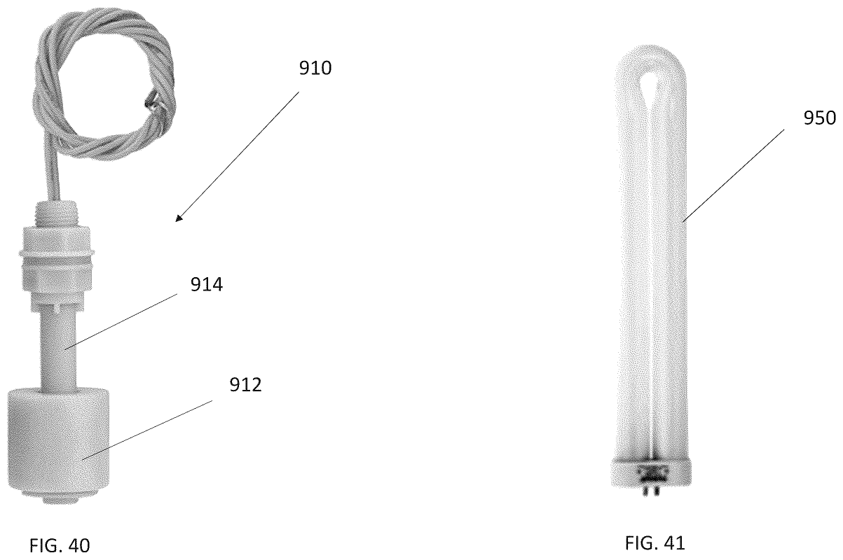

A sensor 910 (shown in FIG. 40) is configured to detect water in the cavity 904. The sensor 910 is a float sensor that includes a float 912 that moves along a stem 914. The float 912 floats on the water in the cavity 904 and moves downward on the stem 914 as a water level in the cavity 904 decreases. A flow of water into the cavity 904 is controlled by a control system (as described above) based on the water level in the cavity 904 detected by the sensor 910. That is, when the float 912 falls to a predetermined position on the stem 914, the control system directs additional water into the cavity 904 through the water inlet 906. As the water enters the cavity 904, the float 912 moves upward on the stem 914. When the float 912 rises to another predetermined position on the stem 914 the control system stops the flow of water into the cavity 904.

An air intake 930 directs air into the cavity 904. An air outlet 932 directs air containing the water droplets out of the cavity 904. The atomizer system 900 is positioned near a fan, for example the fan 840 shown in FIG. 36. In some embodiments, the air outlet 932 is positioned adjacent the fan 840. The fan 840 directs the water droplets from the atomizer system 900 into the hydroxyl ion generator.

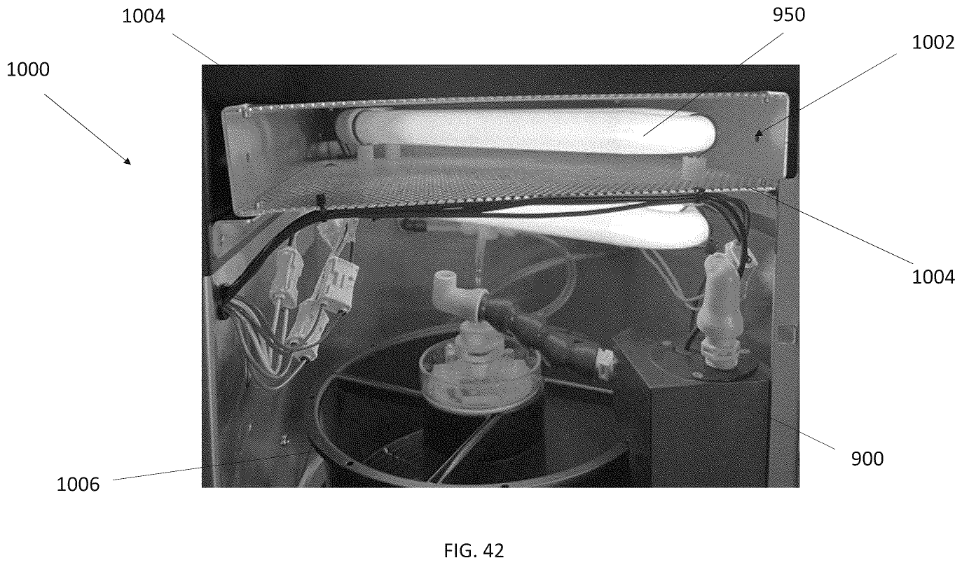

Referring now to FIG. 42, an apparatus 1000 includes a hydroxyl ion generator 1002 having a pair perforated mesh panels 1004 (as described above) and a UV light source 950 (as shown in FIG. 41) positioned between the panels 1004. The atomizer system 900 is positioned below the hydroxyl ion generator 1002. A fan 1006 is configured to direct water droplets generated by the atomizer system 900 into the hydroxyl ion generator 1002, as described in more detail above.

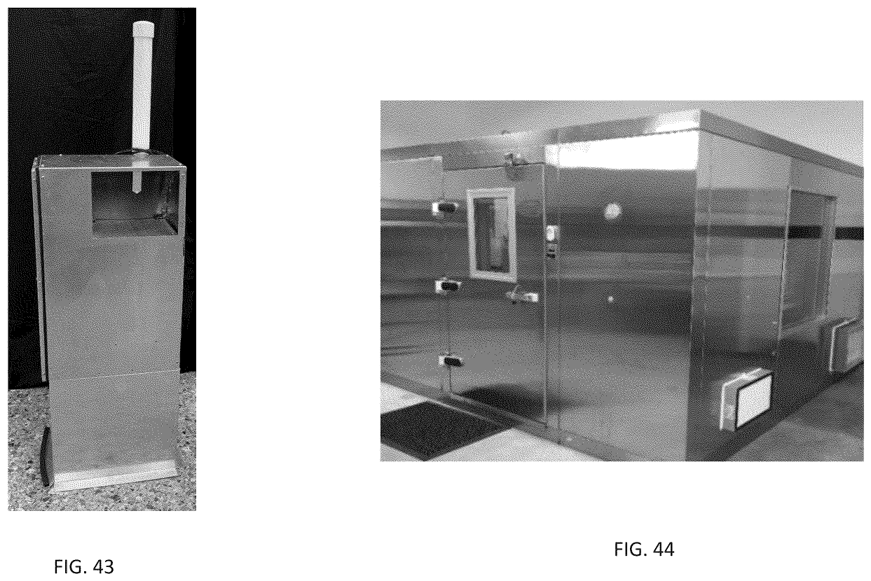

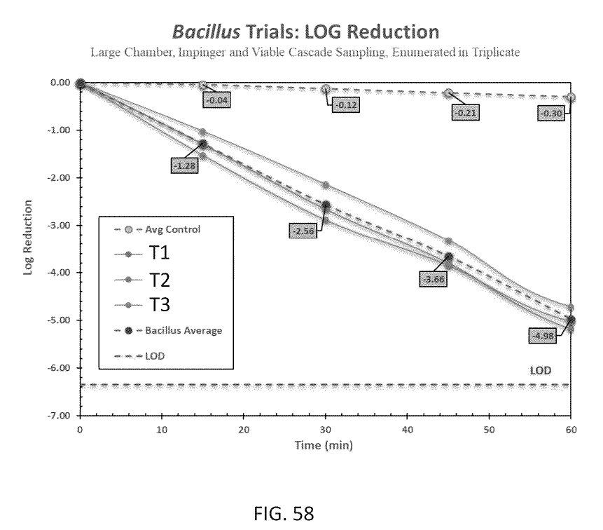

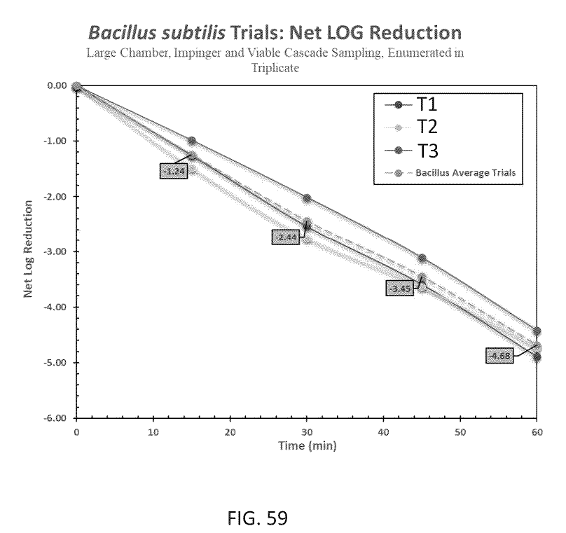

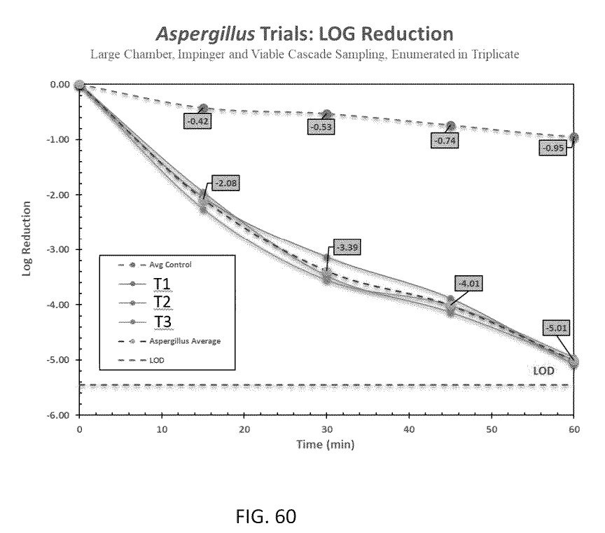

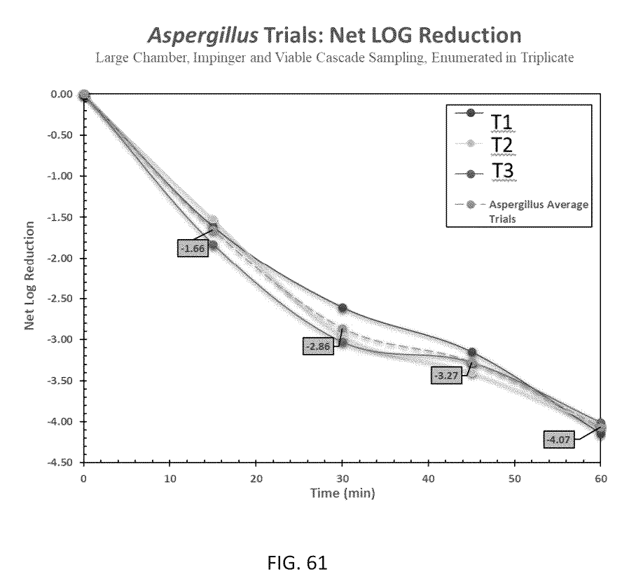

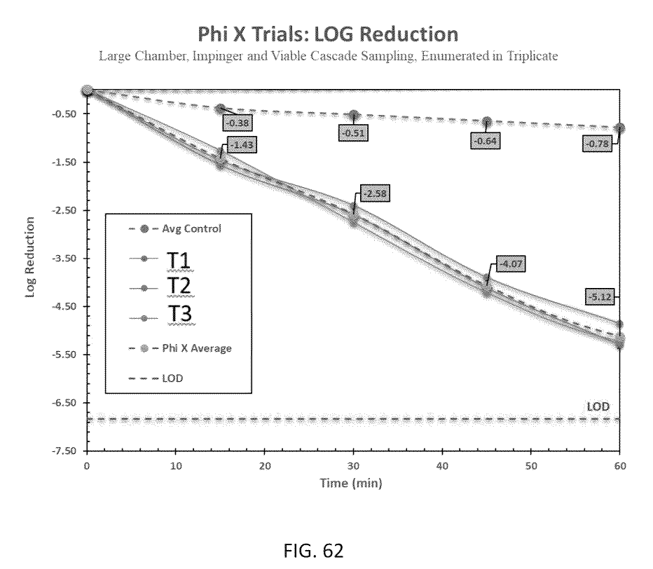

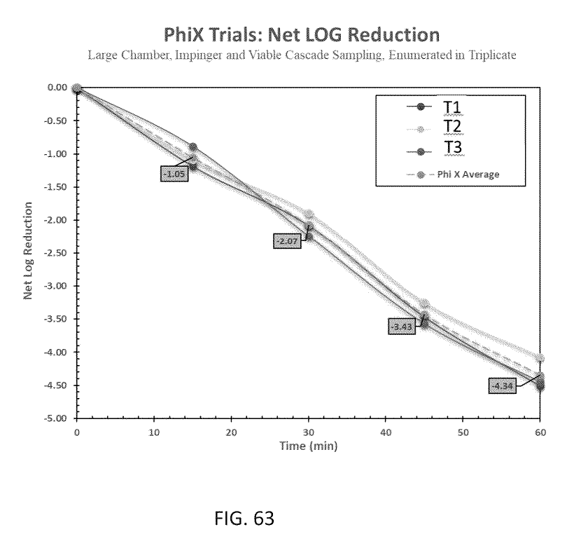

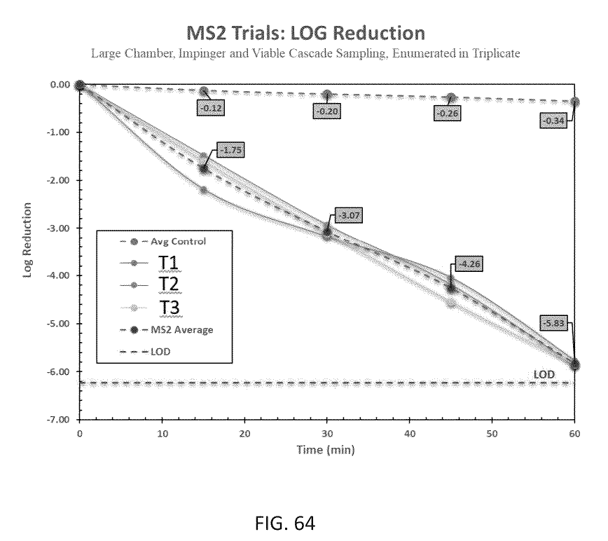

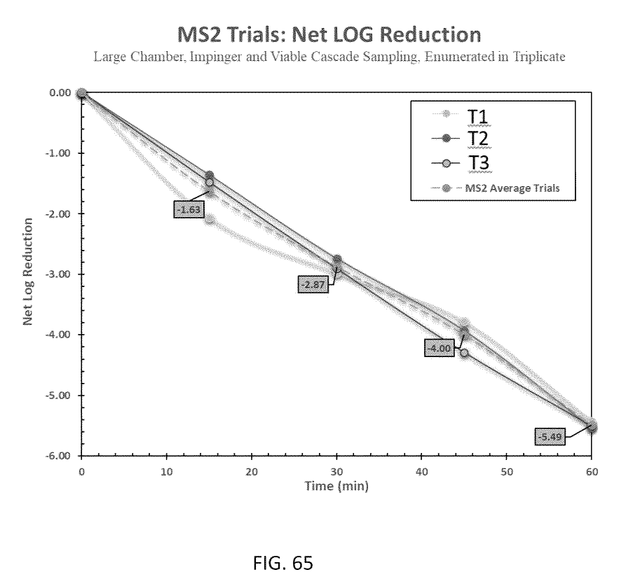

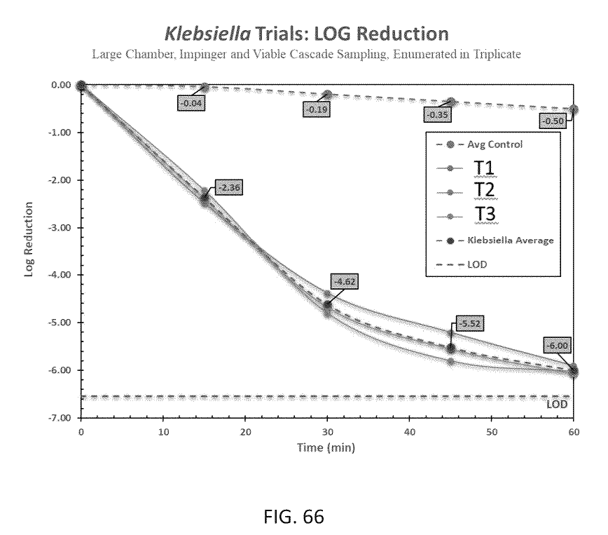

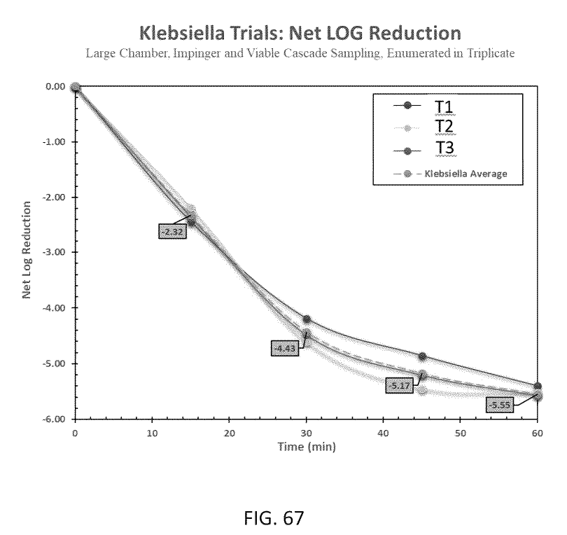

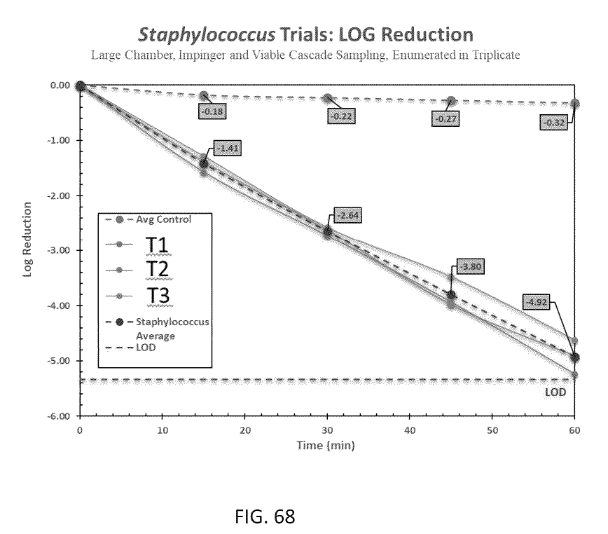

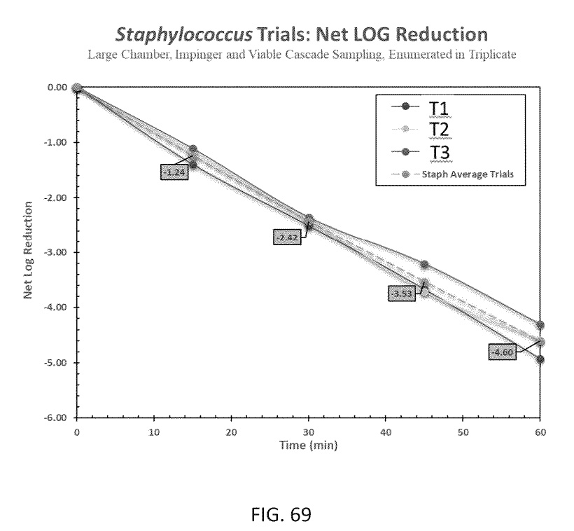

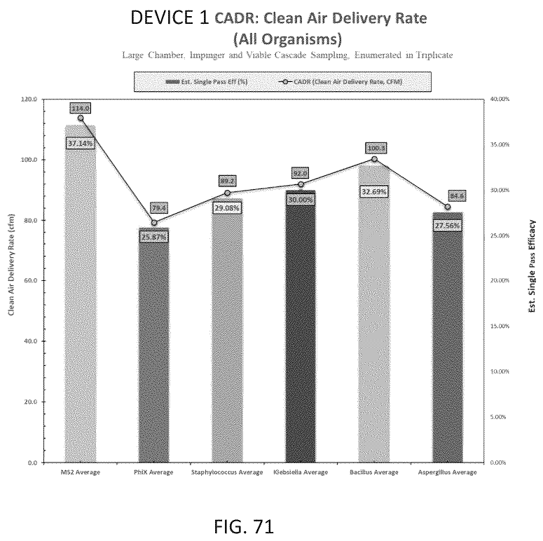

Referring now to FIGS. 43-72, an in-vitro study was performed to characterize the efficacy of an indoor air purifier Test Device for reducing live respirable bioaerosols for six (6) broad-ranged species of microorganisms in a room sized 16 m3 stainless steel bioaerosol test chamber. This study was performed to fulfill the testing requirements for an FDA 510(k) submission.

Background

The Test Device is a free-standing air purifier, equipped with two UV-A lamps housed in the unit with two titanium dioxide (TiO2) coated metal mesh photo-catalyst plates, as well as an air filter. The UV-A lamps, along with the coated mesh, is used to generate hydroxyl radicals that are used to reduce a broad range of gram-positive and gram-negative bacteria, RNA and DNA viruses, bacterial and fungal spores, along with other airborne contaminants in indoor air.

The species selected for this study are recognized surrogates for more dangerous pathogenic organisms. They are 1) MS2, a non-enveloped ssRNA virus, 2) Phi X 174, a non-enveloped ssDNA virus, 3) Klebsiella aerogenes, a gram-negative bacterium, 4) Methicillin Resistant Staphylococcus epidermidis (MRSE), a gram-positive bacterium, 5) Bacillus subtilis bacterial endospores, and 6) Aspergillus brasiliensis spores, one of the most common sources of toxic black mold.

Each microorganism was tested individually and was aerosolized into a sealed 16 m3 environmental bioaerosol chamber, containing the Test Device, using a Collison 24-Jet Nebulizer or dry powder eductor. All the bioaerosols had a mass median aerodynamic diameter (MMAD) ranging from 0.7-4.0 μm (species dependent). Bioaerosol samples were taken at multiple time points throughout each trial, to quantify the reduction rate capability of the air purification device. Impinger samples were serially diluted, plated, incubated, and enumerated in triplicate to yield viable bioaerosol concentration for each sampling point. Chamber control trial data, or natural decay, was subtracted from the device trial data to yield the net log reduction for each of the bioaerosol challenges. Additionally, viable cascades, run at 30 L/min, were used to further resolve the lower detection limits achieved by the Test Device.

Results

The Test Device, operating on it highest fan speed, was effective at reducing the tested bioaerosols by a minimum of 4.0 net log or greater (equivalent to 99.99% reduction) within 60 minutes or less.

Conclusions

The Test Device was shown to be effective at removing bioaerosols from indoor room air. Within 55 min of operation, the device removed >4.0 net log, or 99.99%, of the microorganisms from the 16 m3 test chamber.

The Study

This study was conducted to evaluate the efficacy of the Test Device indoor air purifier at reducing aerosolized microorganisms.

The Test Device device is a free-standing air purifier, equipped with a photocatalytic system comprised of two UV-A lamps and two titanium dioxide (TiO2) photo-catalyst coated plates, and filter housed in the unit. The UV-A lamps and photocatalytic plates are used to create hydroxyl radicals that are intended to reduce a broad range of gram-positive and gram-negative bacteria, RNA and DNA viruses, bacterial and fungal spores, along with other airborne contaminants in indoor air. The Test Device 1 is designed for use in medical facilities, dental offices, classrooms, eldercare facilities, offices, and other indoor spaces.

The test plan incorporated challenging the Test Device with six broad ranging microorganisms, in a closed environmental test chamber, to determine its ability to reduce two separate aerosolized viruses, two separate aerosolized bacteria, and two types of spores. A picture of the Test Device device is shown in FIG. 43. This report will focus on the efficacy of the Test Device for the removal of the broad range of organisms from indoor room air.

Study Overview

The effectiveness of the Test Device was evaluated against an RNA virus, a DNA virus, a gram-negative bacterium, a gram-positive bacterium, a spore forming bacteria, and a mold spore.

Testing was conducted to characterize the reduction efficacy of a single Test Device unit against six aerosolized micro-organisms. This allows for a reasonable demonstration of the capability of the Test Device device to reduce viable bioaerosol concentrations and therefore, theoretically, reducing the chances of exposure to airborne pathogens.

Previous R&D Testing

Previous testing of Test Devices was conducted over the course of several months. Initial testing concluded that the device achieved a high efficiency of bioaerosol removal from the 16 m3 test chamber. Once this initial testing was complete, the testing for all six species was started.

Test Device Description

The Test Device is a standalone air purification system that uses filtration and photocatalysis to generate hydroxyl ions to reduce microorganisms in indoor spaces. The photocatalytic reaction is accomplished with two UV-A lamps and two TiO2 coated metal mesh plates. The UV-A reacts with the coated mesh to produce hydroxyl radicals. It also houses an atomizer that produces water vapor to maintain the humidity within the device for optimal hydroxy creation and reactivity.

The MERV 13 filter, which is used to capture large particles, is followed by the two UV-A lamps and photocatalyst mesh plates. The photocatalyst is used to produce hydroxyl radicals to degrade any biological organisms that may have passed through the filters using the natural reactivity of these radicals.

The device is equipped with a single fan speed and is designed for large spaces. It was placed in the middle of the testing chamber for the duration of all the testing.

Bioaerosol Testing Chamber

A 16 m3 sealed aerosol test chamber, shown in FIG. 44, was used to replicate a contaminated room environment and to contain any potential release of aerosols into the surrounding environment. The aerosol test chamber is constructed with 304 stainless steel and is equipped with three viewing windows and an air-tight lockable chamber door for system setup and general ingress and egress.

The chamber is equipped with filtered HEPA inlets, digital internal temperature and humidity monitor, a heater and humidifier, lighting system, multiple sampling ports, aerosol mixing fans, and a HEPA filtered exhaust system that are operated with wireless remote control. For testing, the chamber is equipped with four ⅜-inch diameter stainless steel probes for aerosol sampling, a 1-inch diameter port for bio aerosol dissemination into the chamber. A Collison 24-jet nebulizer or dry powder eductor are used for the aerosolization of microorganisms and spores, respectively.

To avoid wall effects, all sample and dissemination ports were inserted approximately 18 inches in from the interior walls of the chamber and at a height of approximately 40 inches from the floor. The aerosol sampling and aerosol dissemination probes are stainless steel and bulk headed through the chamber walls to provide external remote access to the aerosol generator and samplers during testing.

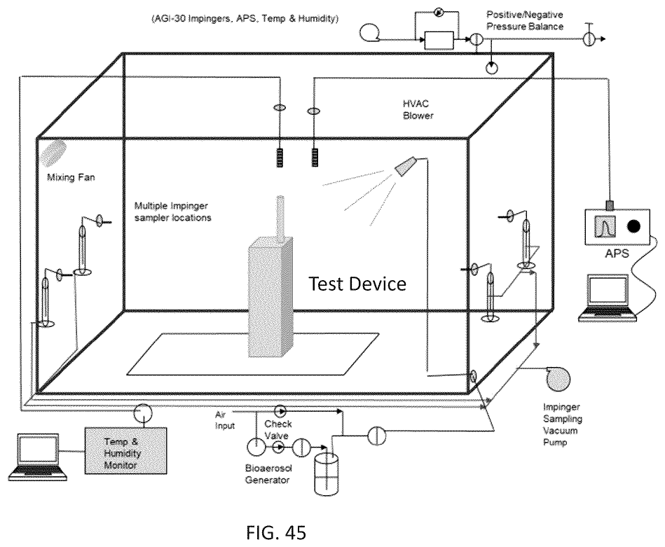

The test chamber is equipped with two high-flow HEPA filters for the introduction of filtered room air into the test chamber during aerosol evacuation/purging of the system between test trials. A HEPA filtered exhaust blower, with a 500 ft3/min rated flow capability, is used for rapid evacuation of remaining bioaerosols. A Magnehelic gauge (Dwyer instruments, Michigan City IN), with a range of −0.5 to 0.5 inches of H2O, is used to monitor and balance the system pressure during aerosol generation, aerosol purge, and testing cycles. A general flow diagram of the aerosol test system is shown in FIG. 45.

Environmental Controls

For increased stability of bioaerosols, the relative humidity inside the chamber was kept consistent at typical indoor levels using a PID humidity controller in combination with an ultra-sonic humidifier to nebulize sterile filtered DI water. Temperature controls maintain chamber trial conditions at typical ambient conditions of 74° F.+/−2° F.

Bioaerosol Generation System

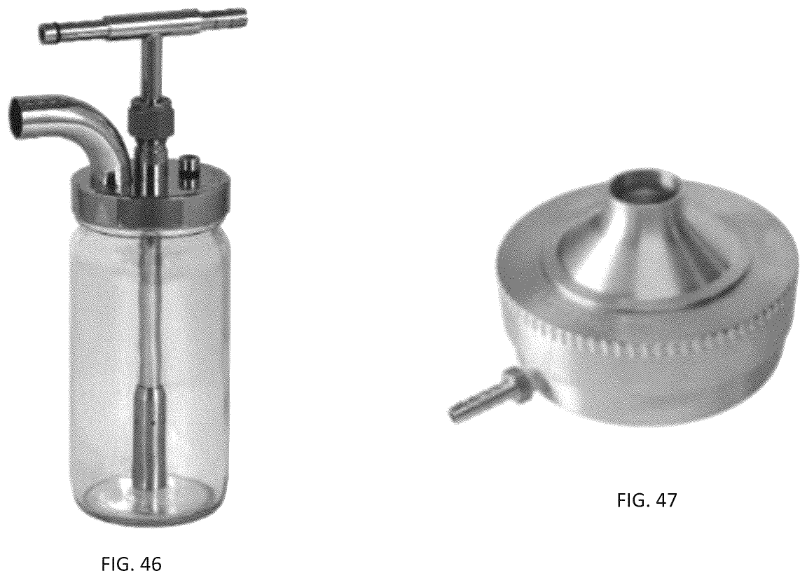

All test bioaerosols were disseminated using a Collison 24-jet nebulizer (BGI Inc. Waltham MA), like the one shown in FIG. 46. The only exception to this is testing with A. brasiliensis spores, which were aerosolized using a dry powder eductor. The aerosolization of bioaerosols was done using a dry, filtered, house air supply. A pressure regulator allowed for control of disseminated particle size, use rate, and sheer force generated within the Collison nebulizer. Prior to testing, the Collison nebulizer flow rate and use rate were characterized using an air supply pressure of approximately 40-60 psi, which produced an output volumetric flow rate of 50-80 L/min with a fluid dissemination rate of approximately 1.25 mL/min. The Collison nebulizer was flow characterized using a calibrated TSI model 4040 mass flow meter (TSI Inc., St Paul MN).

Bioaerosol Sampling and Monitoring System

Two AGI-30 impingers (Ace Glass Inc. Vineland NJ) were used for bioaerosol collection to determine chamber concentrations. The two AGI-30 Impingers were placed at opposite corners of the chamber to represent an entire room sample. The mixing fans inside the chamber worked to ensure a homogenous air mixture inside the chamber.

The AGI-30 impinger vacuum source was maintained at a negative pressure of 18 inches of Hg during all characterization and test sampling to assure critical flow conditions. The AGI-30 impingers sample at a rate of 12.5 LPM impinger flows were characterized using a calibrated TSI model 4040 mass flow meter.

During testing with less resilient organisms, or those which fall out of the air more easily, sample collections were also obtained using a pair of viable cascade impactors. A viable cascade impactor (SKC Inc., Valley View, PA) is comprised of an inlet cone, a precision-drilled 400-hole impactor stage, and a base that holds a standard-size agar plate (FIG. 47).

A high flow pump pulls microorganisms in the air through the holes (jets) at 30 liters per minute, where they are collected directly onto the agar surface. This method is the most sensitive for the detection of organisms at low concentrations.

TSI Aerodynamic Particle Sizer

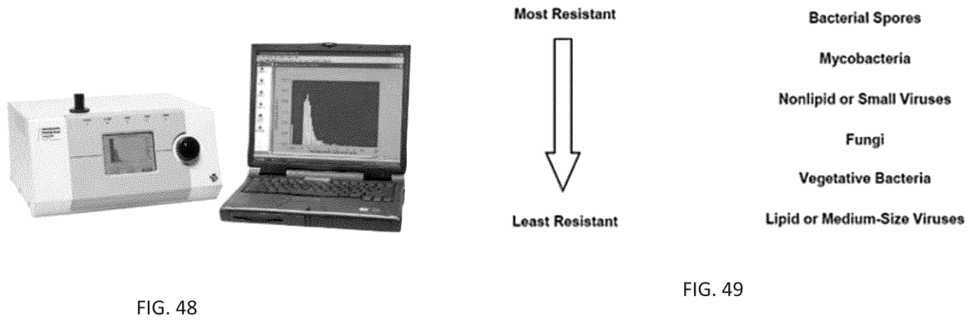

A TSI model 3321 Aerodynamic Particle Sizer (APS) (TSI Inc., Shoreview, MN) was used to measure aerosol concentrations and particle size during the test trials. The APS provided real-time aerodynamic particle characterization with a size range from 0.54-20.0 μm with 52 size bins of resolution. Sampling is continuous with a data export interval of 1 second. The APS has a continuous flow rate of 5 liters per minute (LPM). A picture of the APS is shown in FIG. 48.

Species Selection

Due to safety concerns for bioaerosol testing, organism selection was based on Biological Safety Level 1 (BSL1) species which served as surrogates for more dangerous pathogenic (BSL2 & BSL3) organisms.

The species selected for this study are recognized as surrogates for more dangerous pathogenic organisms. In this study, the species tested were;
1) MS2, a non-enveloped ssRNA virus that is a common surrogate for influenza viruses and is a tentative surrogate for SARS-CoV-2.
2) Phi X 174, a non-enveloped ssDNA virus, that is a surrogate for herpes simplex and smallpox viruses.
3) Klebsiella aerogenes, a gram-negative bacterium used as a surrogate for other Klebsiella infections, a common infectious pathogen that can cause pneumonia, bloodstream, and surgical wound infections, in addition to meningitis.
4) Methicillin Resistant Staphylococcus epidermidis (MRSE), a gram-positive bacterium that serves as a surrogate for many pathogenic gram-positive bacteria, including Methicillin Resistant Staphylococcus aureus (MRSA).
5) Bacillus subtilis bacterial endospores, which serve as a model for the many bacterial species that produce highly resistant endospores such as Anthrax (Bacillus anthracis).
6) The spores from Aspergillus brasiliensis, one of the most common sources of toxic black mold.

Viral Challenges

MS2 bacteriophage is a viral single-stranded, non-enveloped RNA bacteriophage that has historically been used as a surrogate for influenza viruses. MS2 has also recently been used as a tentative surrogate for SARS-CoV-2 in numerous published bioaerosol studies. PhiX-174 (Φ-X174) is a viral, single-stranded, non-enveloped, DNA bacteriophage traditionally used as a surrogate for viral species such as herpes simplex and smallpox.

The US FDA guidance document, Enforcement Policy for Sterilizers, Disinfectant Devices, and Air Purifiers During the Coronavirus Disease 2019 (COVID-19) Public Health Emergency, states that lipid enveloped viruses, such as coronaviruses, are the least resistant microorganisms to germicidal chemicals. It is presumed that this susceptibility is similar for other chemical, physical, and catalytic methods of destruction.

MS2 and Phi X174 are non-enveloped viruses, which makes them more resistant to disinfection than lipid viruses, and therefore, should represent a “worst case scenario” when compared to actual lipid-enveloped RNA viruses like SARS-CoV-2. FIG. 49 is a graphic from the FDA document, COVID Sterilizers, Disinfectant Devices, and Air Purifiers Guidance, demonstrating resistance to disinfection.

These results were obtained by investigations on many different coronaviruses, including SARS-CoV and MERS-CoV, but not SARS-CoV-2. Nevertheless, it can be assumed that they are also applicable for SARS-CoV-2 and all future mutations. RNA mutations might have a strong influence on the pathogenicity of a virus, but they do not result in larger structural differences, especially concerning the UV absorption properties of the RNA, which are the main cause for the antiviral effect of ultraviolet radiation.

Vegetative Bacteria Challenges

The vegetative bacteria organisms used for this study included Methicillin Resistant Staphylococcus epidermidis (MRSE) (ATCC 12228). Staphylococcus epidermidis is a gram-positive bacterium and BSL1 simulant for a wider range of medically significant pathogens including Methicillin Resistant Staphylococcus aureus (MRSA). Klebsiella aerogenes is a gram-negative bacterium, and a BSL1 surrogate for other Klebsiella infections, which is a commonly found food borne pathogen.

Mold Spores and Bacterial Endospore Challenges

Aspergillus brasiliensis (ATCC 16404), formerly known as A. niger, is one of the most common species of the genus Aspergillus. A. brasiliensis is routinely defined as a surrogate for various toxic black mold species such as Stachybotrys chartarum. Many respiratory problems found in infants, the elderly, and immunocompromised individuals are attributed to mold. Purified A. brasiliensis spores were used in bulk, dry powder form with an approximate concentration of 1×109 cfu/gram.

Bacillus subtilis (ATCC 49760), endospores were used as a surrogate for Bacillus anthracis (Anthrax), a biological agent used for bioterrorism/bio warfare research. It also serves as a surrogate for other pathogenic endospore forming species such as Clostridioides difficile, a common and difficult to eliminate hospital pathogen. Bacillus subtilis, a sub-species of Bacillus atrophaeus, is a gram-positive bacterium found in soil and in the gastrointestinal tract of ruminants and humans. B. subtilis is rod-shaped, and forms a tough, highly resistant endospore, which allows it to tolerate extreme environmental conditions.

Bioaerosol Challenge Particle Size Testing

Bioaerosol challenge particle size distributions were measured with a TSI Aerodynamic Particle Sizer model 3321 (APS) for all challenge species. The particle size distribution was taken shortly after aerosolization for each species via sampling through a sample probe into the test chamber. The APS has a dynamic measurement range of 0.54 to 20.0 μm and was programmed to take consecutive real-time one-minute aerosol samples. Data was logged in real-time to an Acer laptop computer, regressed, and plotted.

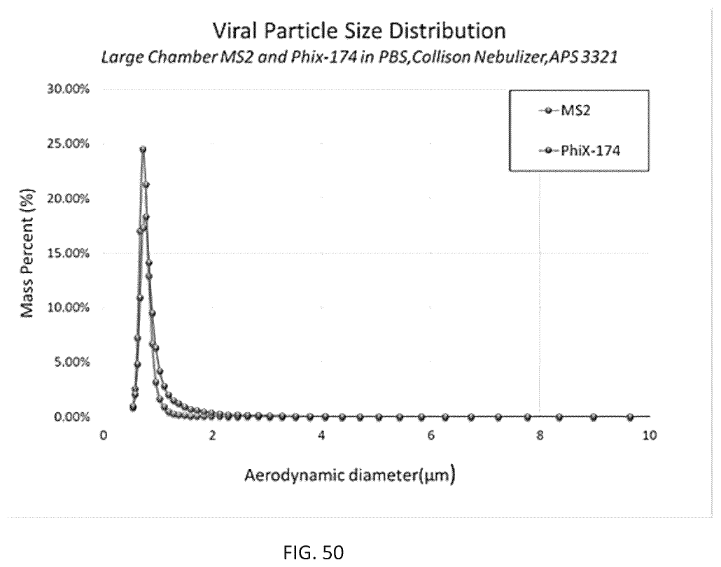

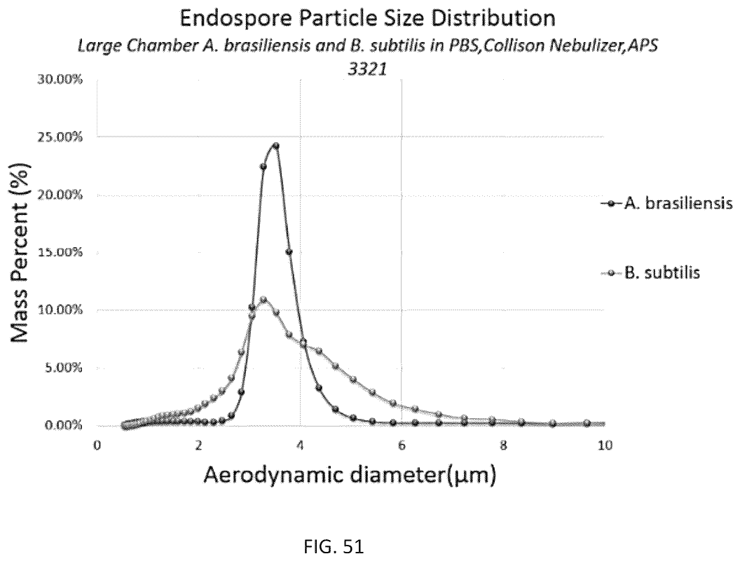

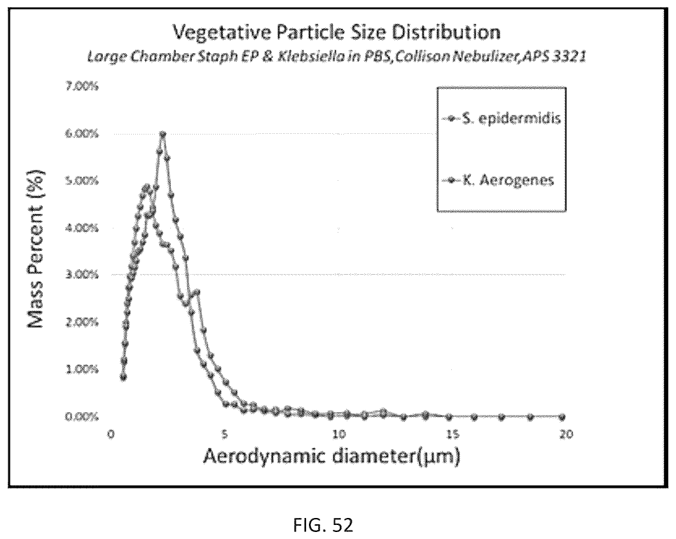

The aerodynamic particle size distribution for all challenge bioaerosols is shown to be within the respirable range for regional alveolar tract deposition and show a low geometric standard deviation (GSD), indicating that a monodispersed aerosol was generated in the chamber for each of the challenge species. The aerodynamic particle size distributions for MS2 and Phi X174 can be found in FIG. 50, for A. brasiliensis, B. subtillis in FIG. 51, and K. aerogenes, and S. epidermidis in FIG. 52.

While there may be variation in the particle size of a single bacteria or virus to the dispersal size, shown in the previously mentioned graphs, any virus that is encountered in nature is most likely suspended in a matrix solution. While viruses may be smaller than the particle size shown these aerosolized particles are far more representative of a real-life situation where the device would encounter these organisms.

Viral Culture & Preparation

Pure strain viral seed stock and host bacterium were obtained from ATCC. Host bacterium was grown in a similar fashion to vegetative cells in an appropriate liquid media. The liquid media was infected during the logarithmic growth cycle with the specific bacteriophage.

After an appropriate incubation time, the cells were lysed, and the cellular debris separated by centrifugation. MS2 stock yields were greater than 1×1011 plaque forming units per milliliter (pfu/mL) with a single amplification procedure. This stock MS2 viral solution was then diluted with PBS to approximately 1×1010 plaque forming units per milliliter (pfu/mL) for use in the Collision nebulizer. The Phi X174 stock was prepared in the same manner however, to achieve a high enough concentration, the Phi X 174 underwent a double amplification procedure.

Vegetative Cells Culture & Preparation

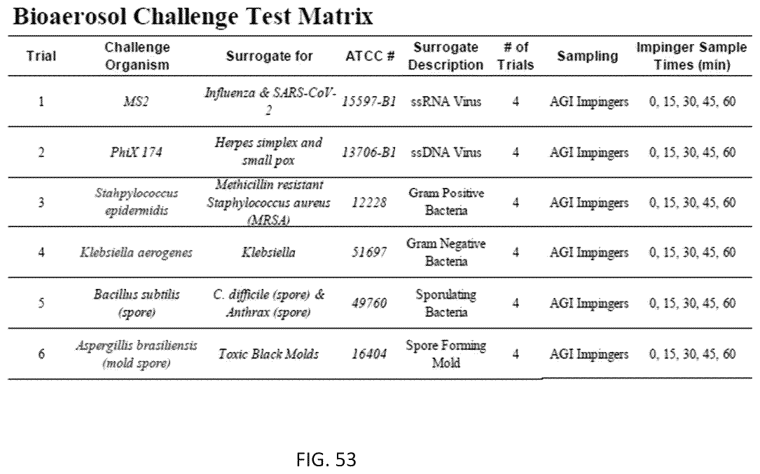

Pure strain seed stocks were purchased from ATCC (American Type Culture Collection, Manassas VA). For ATCC reference numbers see FIG. 53.

Working stock cultures were prepared using aseptic techniques in a class 2 biological safety cabinet and followed standard preparation methodologies. Approximately 250 mL of each biological stock was prepared in tryptic soy liquid broth media, and incubated for 24-48 hours with oxygen infusion (1 cc/min) at 37° C. Biological stock concentrations were around 1×1010 cfu/ml.

Stock cultures were centrifuged for 10 minutes at 3000 rpm in an LD-3 centrifuge in sterile 15 mL conical tubes, growth media was removed, and the cells re-suspended in sterile PBS buffer for aerosolization.

Aliquots of these suspensions were enumerated on tryptic soy agar plates (Hardy Diagnostics, Cincinnati OH) for viable counts and stock concentration calculation. For each organism, test working stocks were grown in sufficient volume to satisfy use quantities for all tests conducted using the same culture stock material.

Fungal Spore Culture & Preparation

Aspergillus brasiliensis fungal spores were obtained in purified bulk powder form at a concentration of 1×109 cfu/g. To verify the bulk powder spore concentration, an aliquot of weighed dry powder was prepared in suspension in PBS+0.005% Tween 80 at a mass:volume ratio to obtain a concentration of 1×109 cfu/ml. This aliquoted spore suspension was plated prior to testing to verify concentration.

Bacillus subtilis freeze-dried spores were purchased from ATCC with a stock concentration of 1×1011 cfu/gram. One gram of dry spores was suspended in a 250 mL solution of 50/50 91% Isopropyl alcohol and PBS+5% Tween to assist in de-agglomeration. This suspension was sonicated for 40 minutes to bring all powder into solution. This aliquoted spore suspension was plated prior to testing to verify concentration. Aliquots of these suspensions were enumerated on tryptic soy agar plates (Hardy Diagnostics, Cincinnati OH) for viable counts and stock concentration calculation. For each organism, test working stocks were grown in sufficient volume to satisfy use quantities for all tests conducted using the same culture stock material.

Fungal Spore Culture & Preparation Aspergillus brasiliensis fungal spores were obtained in purified bulk powder form at a concentration of 1×109 cfu/g.

To verify the bulk powder spore concentration, an aliquot of weighed dry powder was prepared in suspension in PBS+0.005% Tween 80 at a mass:volume ratio to obtain a concentration of 1×109 cfu/ml. This aliquoted spore suspension was plated prior to testing to verify concentration.

Bacillus subtilis freeze-dried spores were purchased from ATCC with a stock concentration of 1×1011 cfu/gram. One gram of dry spores was suspended in a 250 mL solution of 50/50 91% Isopropyl alcohol and PBS+5% Tween to assist in de-agglomeration. This suspension was sonicated for 40 minutes to bring all powder into solution. This aliquoted spore suspension was plated prior to testing to verify concentration.

Plating and Enumeration

Impinger and stock biological cultures were serially diluted and plated in triplicate. (Multiple serial dilutions) using a standard spread plate assay technique onto tryptic soy agar plates. The plated cultures were incubated for 24-48 hours, depending on the species, then enumerated and recorded.

When working with microorganisms, at extremely low concentrations, viable cascade sampling was used. This method samples the chamber by pulling 30 liters per minute through the cascade device directly onto an agar plate. Enumeration of colonies grown depends on the concentration of the sample. Colony counts, totaling up to 400, can then be adjusted using the positive conversion table. This table is based on the principle that, as the number of viable particles being impinged on a given plate increases, the probability of the next particle going into an “empty hole” decrease. This can be corrected statistically using the conversion formula of Feller, W (1950).

Methods: Bioaerosol Efficacy Testing

To accurately assess the Test Device, test chamber pilot control trials were performed with all organisms over a 120-minute period to characterize the biological challenge aerosol delivery/collection efficiency, and viable concentration over time. Control testing was performed to provide baseline comparative data to assess the actual reduction from the Test Device challenge testing and verify that viable bioaerosol concentrations persisted above the required concentrations over the entire pilot control test period.

During control runs, a single low velocity fan, located in the corner of the bioaerosol test chamber was turned on for the duration of the trial to ensure a homogenous aerosol concentration within the aerosol chamber. The mixing fan was used for all control runs and was turned off during Test Device decontamination trials. The two impingers used for bioaerosol collection were pooled and mixed prior to plating and enumeration. A complete test matrix for the bioaerosol trials can be found in FIG. 53.

For each control and challenge test, the Collison nebulizer was filled with approximately 40 mL of biological stock and operated at 40 psi for a period of 20 minutes. Then, the impingers were filled with 20 mL of sterilized PBS with an addition of 0.005% v/v Tween 80 for bioaerosol collection. The addition of Tween 80 was used to increase the impinger collection efficiency and de-agglomeration of all microorganisms. The chamber mixing fan was turned on during bioaerosol dissemination to assure a homogeneous bioaerosol concentration in the test chamber prior to taking the first impinger sample (T=0).

Following bioaerosol generation, baseline bioaerosol concentrations were established for each pilot control and Test Device test by sampling simultaneously with two AGI-30 impingers located at opposite corners of the chamber. The samples were collected for 3 to 20 minutes at intervals of 15 or 30 minutes throughout the entire test period.

Collected impinger chamber samples were pooled and mixed at each sample interval for each test. Aliquots of impinger samples were collected and then used for plating. Impingers were rinsed 6× with sterile filtered water between each sampling interval and re-filled with sterile PBS using sterile graduated pipettes for sample collection. During the final 2-3 time points, a viable cascade was used to further increase the limits of detection for the testing.

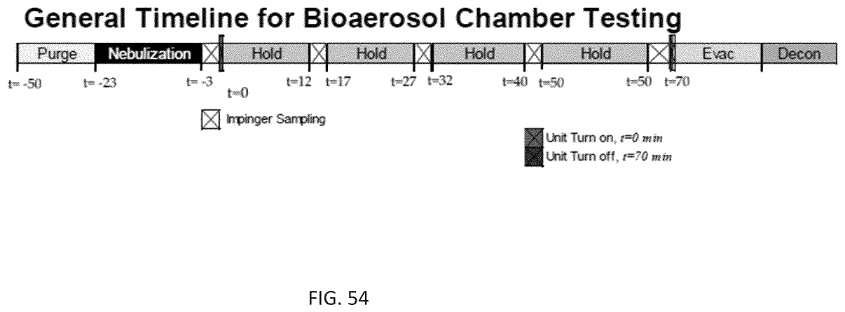

For Test Device biological testing, the unit was turned on immediately following a time 0 (T=0) baseline sample and operated for the entirety of the test. Subsequent impinger samples were taken at various time points throughout the trial. These samples were enumerated for viable concentration to measure the effective viable bioaerosol reduction during operation of the Test Device over time. See FIG. 54 for a timeline of the test trials.

All samples were plated in triplicate on tryptic soy agar media over a minimum 3-log dilution range. Plates were incubated for 24-48 hours and enumerated for viable plaque forming units (pfu) or colony forming units (cfu) to calculate aerosol challenge concentrations in the chamber and reduction of viable microorganisms.

Post-Testing Decontamination and Prep

Following each test, the chamber was air flow evacuated/purged for a minimum of twenty minutes between tests and analyzed with the APS for particle concentration decrease to baseline levels between each test. The chamber was decontaminated at the conclusion of the trials with aerosol/vaporous hydrogen peroxide (35%). The Collison nebulizer and impingers were cleaned at the conclusion of each day of testing by soaking in a 3% bleach bath for 20 minutes. The nebulizer and impingers were then submerged in a DI water bath, removed, and spray rinsed 6× with filtered DI water until use.

Data Analysis

Results from the control trials were graphed and plotted to show natural viability loss over time in the chamber. These control runs served as the basis to determine the time required for the Test Device to achieve at least a 4 LOG (99.99%) reduction in viable bioaerosol above the natural losses from the control runs. The control and trial runs are plotted showing log reduction in viable bioaerosol for each organism. All data is normalized with time zero enumerated concentrations. Subsequent samples are normalized and plotted to show the loss of viability over time.

Test Results

This study was performed to evaluate the Test Device's efficacy for the reduction of bioaerosols in an environmental test chamber. Reduction of viable bioaerosols by 4 logs, or 99.99%, is the minimum requirement for FDA approved use. The microorganism species were selected specifically for their ability to gauge the device's efficacy for reducing the most frequently encountered organisms. Greater than a 4 net log reduction was achieved for all the organisms that were tested.

For the MS2 bacteriophage, the device showed a maximum net log reduction of 5.18+/−0.08 in 60 minutes while reaching 4 net log reduction in approximately 40 minutes, and for Phi X 174, the device achieved a maximum net log reduction of 4.34+/−0.24 in 60 minutes and hitting the 4.0 net log reduction goal in just 55 minutes.

The first bacteria tested, Staphylococcus epidermidis, reached a maximum net log reduction of 4.58+/−0.31 in 60 minutes, and 4.0 net log reduction in just over 50 minutes.

The second bacteria tested, K. aerogenes, had the highest reduction reaching a 5.55+/−0.09 maximum net log reduction in 60 minutes and 4.0 net log reduction in 25 minutes.

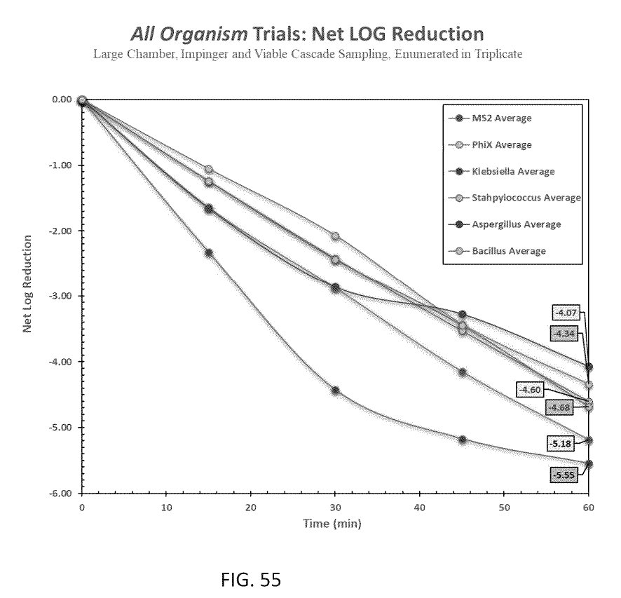

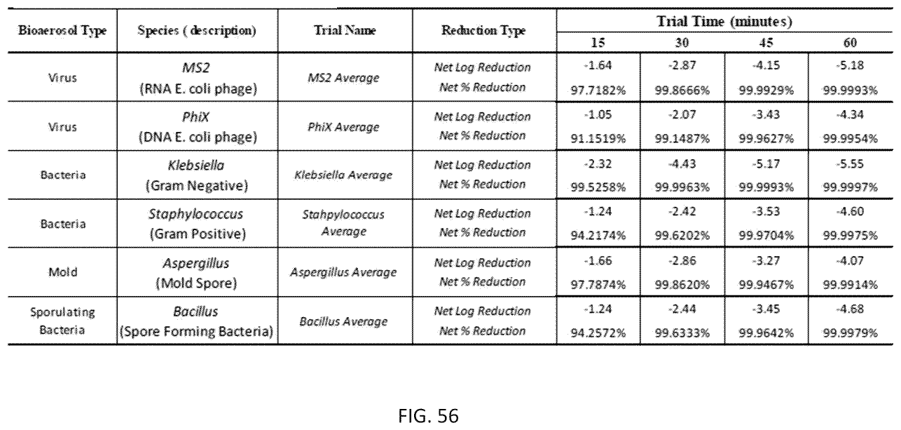

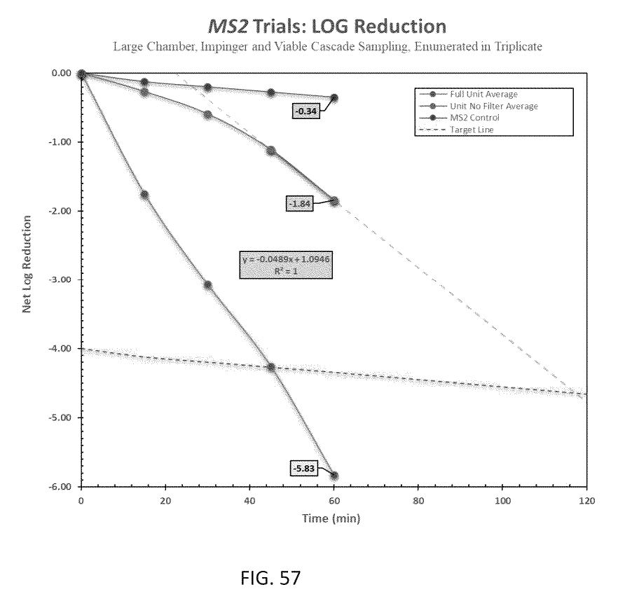

Aspergillus brasiliensis mold spores showed an average net reduction of 4.07+/−0.07 net log in 60 minutes and reached 4.0 net log reduction just before that time. The bacterial endospore from Bacillus subtilis reached a net log reduction of 4.68+/−0.24 in 60 minutes while reaching the 4.0 net log reduction in 50 minutes. A plot of the net log reduction data can be found in FIG. 55 and is summarized in FIG. 56.

Deviations and Acceptance Criteria

No deviations from the protocol were noted throughout the test trials. All final endpoints were ≤0.30 standard deviations from the mean. In accordance with ARE Labs standard practices, and in compliance with GLPs, all data was verified for accuracy.

Conclusion

In conclusion, the Test Device device achieved >4 net log reduction of all bioaerosols within 25 to 60 minutes. The device proved to be highly effective at reducing the viability of a broad range of aerosolized microbial species.

It is anticipated that such a reduction should reduce the likelihood of individuals being exposed to and contracting airborne infectious diseases in an enclosed environment, medical or otherwise. Given a higher starting concentration and longer test trials, it is safe to assume that a 6 net log reduction is achievable for all of the organisms.

One of the main mechanisms of bioaerosol reduction for the Test Device is the production of hydroxyl radicals. However, measuring the actual production of hydroxyl radicals directly is quite difficult. To contend with this, an indirect method was utilized instead to measure the effect of the ions on MS2 rather than measuring the actual quantity of ions produced.

The main mechanisms of bioaerosol reduction are the filter and hydroxyl radical production. By removing the filter and then running test trials, a comparison can be made with the complete device, with filter installed, and the effect hydroxyl radicals on the overall reduction capability of the device. While the filter serves as a major factor in bioaerosol reduction, the hydroxyl production also serves a significant role. Based on the results seen in FIGS. 57-69, the reduction achieved, with no filter installed, accounted for approximately 27% of the total reduction achieved by the fully functioning device.

One thing to note about this however is that the trend for the total device reduction is fairly linear while the reduction with no filter at all seems to have more of a curved downward trend. This makes sense given the logic that the accumulation of these hydroxyl radicals would have a more compounding effect on reduction over time. With a linear trendline applied to the last two data points of the trial sets, run without filters, an extrapolation can be made of for the possible reduction achieved over a certain amount of time with hydroxyl radical accumulation. When the trial time is doubled to 120 min, based on the trendline equation, the reduction provided just by the hydroxyl production would be approximately a 4.77 log not factoring in control loses.

Given this information and the extrapolation of the trendlines seen based on the data, the accumulation of hydroxyl radical production would have a significant impact on bioaerosol reduction. The filter allows for the Test Device to remove bioaerosols quickly while the hydroxyl production accumulation provides a more long-term impact on the reduction potential.

Calculations

To evaluate the viable aerosol delivery efficiency and define operation parameters of the system, calculations based on (theoretical) 100% efficacy of aerosol dissemination were derived using the following steps:
Plating and enumeration of the biological to derive the concentration of the stock suspension (Cs) in pfu/mL or cfu/mL, or cfu/g for dry powder.
Collison 24 jet nebulizer use rate (Rneb) (volume of liquid generated by the nebulizer/time) at 28 psi air supply pressure=1.0 mL/min.
Collison 24 jet Generation time (t)=20 or 30 minutes, test dependent.
Chamber volume (Vc)=15,993 Liters

Assuming 100% efficiency, the quantity of aerosolized viable particles (VP) per liter of air in the chamber for a given nebulizer stock concentration (Cs) is calculated as:

$$Nebulizer : {V}_{P} = \frac{{C}_{s} \cdot {R}_{neb}}{{V}_{c}} t$$

Plating and enumeration of the biological to derive the concentration of the dry powder (Cp) in cfu/g.
Eductor use rate (M p) (Mass of powder generated by the eductor in grams)
Chamber volume (Vc)=15,993 Liters

Assuming 100% efficiency, the quantity of aerosolized viable particles (VP) per liter of air in the chamber for a given dry powder stock concentration (Cp) is calculated as:

$$Eductor : {V}_{P} = \frac{{C}_{p} \cdot {M}_{p}}{{V}_{c}}$$

AGI—30 impinger or 47 mm filter collection calculation:
Viable aerosol concentration collection (Ca)=cfu or pfu/L of chamber air.
Viable Impinger concentration collection (CImp)=cfu or pfu/mL from enumeration of impinger sample or filter sample.
Impinger sample collection volume (Ivol)=20 mL collection fluid/impinger, or extraction fluid for filter.
AGI-30 impinger or filter sample flow rate (Qimp)=12.5 L/min.
AGI-30 impinger or filter sample time (t)=5 or 10 minutes, test dependent.

For viable impinger or filter aerosol concentration collection (Ca)=cfu or pfu/L of chamber air:

$${C}_{a} = \frac{{C}_{Imp} \cdot {I}_{vol}}{{Q}_{imp}} t$$

The aerosol system viable delivery efficiency (expressed as %) is:

$$Efficiency = \frac{{C}_{s}}{{V}_{p}} \cdot 100$$

Clean Air Delivery Rate (CADR)

In addition to testing, the clean air delivery rate (CADR) was calculated for the Violett M. The clean air delivery rate is the air flow from the device that has been purified of particles in each size distribution. This is calculated by multiplying the efficiency of the device by its flow rate in cubic feet per minute (CFM).

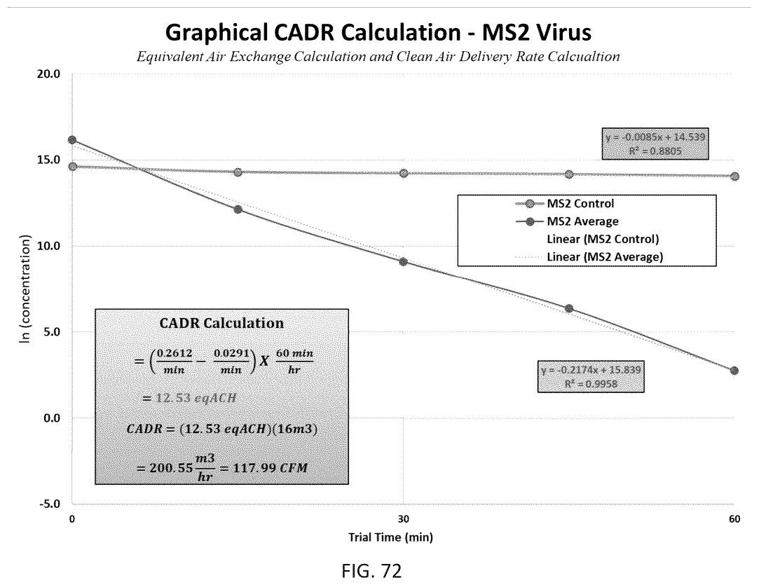

For CADR calculations, the difference in slopes for the control and test trials was calculated to determine the equivalent air exchange rate. The slope of the test trials was determined using only the T-0 and T-20 time points due to low bioaerosol concentrations within the chamber that were close to limits of detection past this timepoint. The CADR was then calculated by multiplying the equivalent air exchange rate by the volume of the test chamber (16 m3). See FIG. 72 for an example of these calculations.

Clean Air Delivery Rate (CADR) Results

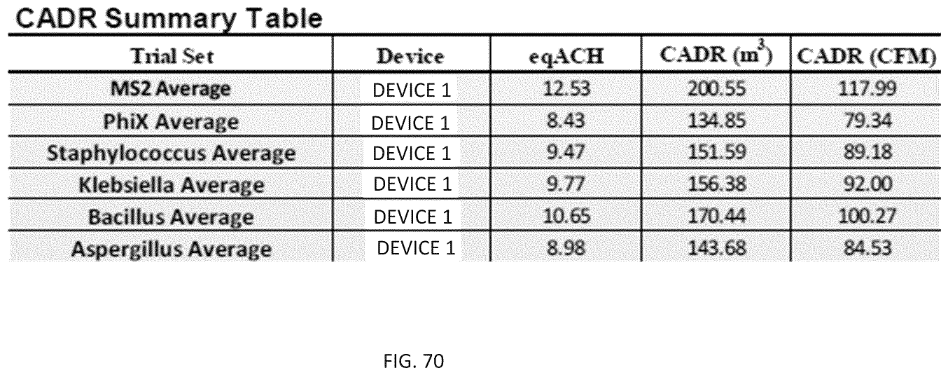

The calculated CADR, based off trial data, suggests that Test Device can achieve a range of 8.43 to 12.53 equivalent air exchanges (eqACH) per hour depending on the organism. A table breakdown of these results is shown in FIG. 70. While this number is only an estimation of the device's ability to remove viruses from a given volume, it does provide a general assessment of the device's performance in the bioaerosol test chamber.

The device is easily able to reach the 4-log reduction threshold with a CADR of between 134.85 and 200.55 m3 which translates to a 79.34 to 117.99 CADR in CFM (cubic feet per minute). All these values average to an equivalent air exchange of 9.90 per hour, or the theoretical number of turnovers of clean air simulated by the device in a single hour across all organisms.

Mold Test

A mold test was performed to compare the effectiveness of the devices described herein against mold growth on raspberries with two different UV light bulb types. The test also compared the effectiveness that a titanium dioxide coated plate adds to the effectiveness of each bulb. During the test five containers were used to test 5 different variants against mold growing on raspberries. The raspberries were washed and dried and 8-12 raspberries were placed on a paper towel square inside each container of the five containers. In the first container, the control, there were only raspberries. In the second container, there were raspberries and the system was equipped with a Damar® #00548H bulb available from Damar Worldwide, having an address of 805 N Carnation Dr, Aurora, MO 65605. In the third container, there were raspberries and the system was equipped with a Damar® #00548H bulb and a titanium dioxide coated plate. In the fourth container, there were raspberries and the system was equipped with a Damar® #01703B bulb. In the fifth container, there were raspberries and the system was equipped with a Damar® #01703B bulb and a titanium dioxide coated plate.

The control started to grow mold on day 1. Container 5 did not grow mold until day 8. After 12 days, all of the containers contained mold, however Container 5 only had white mold spores that had popped, but not spread. The control had molds that were white, green, black and yellow. Container 2 had molds that were white, green and yellow. Container 3 had white, green and a little white mold. Container 4 had black and white mold. Container 5 only had white mold. Accordingly, in some embodiments, the systems described herein are equipped with a Damar® #01703B bulb. Additionally, in some embodiments, the systems described herein are also equipped with a titanium dioxide coated plate.

Although the present invention has been described with reference to preferred embodiments, persons skilled in the art will recognize that changes may be made in form and detail without departing from the spirit and scope of the invention. All references cited throughout the specification, including those in the background, are incorporated herein in their entirety. Those skilled in the art will recognize, or be able to ascertain, using no more than routine experimentation, many equivalents to specific embodiments of the invention described specifically herein. Such equivalents are intended to be encompassed in the scope of the following claims.

The following clauses enumerated consecutively from 1 through 210 provide for various aspects of the present invention. In one embodiment, in a first clause (1), the present invention provides an apparatus comprising:
a portal comprising at least one opening positioned on a panel of the portal; and
a hydroxyl ion generator in communication with the portal and the at least one opening, wherein hydroxyl ions can be transmitted through the at least one opening to emit the hydroxyl ions into the area formed by the portal.

2. The apparatus of clause 1, wherein the panel includes first and second opposing side panels, wherein a first hydroxyl ion generator is in communication with the first side of the portal.

3. The apparatus of clause 2, wherein a second hydroxyl ion generator is in communication with the second side of the portal.

4. The apparatus of clause 3, further comprising at least one fan in communication with either the first or second hydroxyl ion generator.

5. The apparatus of clause 2, further comprising a top portion of the portal in communication with the first and second opposing side panels of the portal.

6. The apparatus of clause 5, wherein the top portion has an arched configuration.

7. The apparatus of clause 5, wherein the top portion and the first and second opposing side panels are integrally formed.

8. The apparatus of clause 5, wherein the portal is positioned on a surface so that the first and second opposing side panels extend from the surface, wherein the top portion is positioned opposite the surface.

9. The apparatus of clause 8, wherein the top portion, the surface, and the first and second opposing side panels form a rectangular configuration.

10. The apparatus of clause 8, wherein the top portion is spaced apart from the surface so that a person can pass between the top portion and the surface.

11. The apparatus of clause 8, wherein the top portion is spaced at least one inch from the surface.

12. The apparatus of clause 8, wherein the top portion, the surface, and the first and second opposing side panels form a trapezoidal configuration.

13. The apparatus of clause 5, wherein the top portion further comprises at least one top portion opening, wherein the hydroxyl ions can be transmitted through the at least one top portion opening.

14. The apparatus of clause 3, further comprising at least one fan in communication with the first hydroxyl ion generator and at least one fan in communication with the second hydroxyl ion generator.

15. The apparatus of clause 1, wherein the hydroxyl ion generator comprises a perforated mesh coated with titanium dioxide, a water source, a humidification system and a UV light generator that provides a spectrum of light from about 320 nm to about 385 nm.

16. The apparatus of clause 15, wherein the water source is a continuous water source.

17. The apparatus of clause 15, wherein the water source includes water stored in a container.

18. The apparatus of clause 17, further comprising an attached float to regulate the level of water in the container.

19. The apparatus of clause 15, wherein the humidification system is an ultrasonic humidification system.

20. The apparatus of clause 15, further comprising a water filter in communication with the water source and the humidification system.

21. The apparatus of clause 1, further comprising a conveyor system in communication with the portal, wherein objects can be passed through the portal system on the conveyor system.

22. The apparatus of clause 2, wherein the first and second opposing side panels are spaced at least one inch apart.

23. The apparatus of clause 1, wherein the panel defines an opening of at least one inch in at least one dimension, wherein the hydroxyl ions are emitted into the opening.

24. The apparatus of clause 1, wherein the panel forms a circular configuration having an opening, wherein the hydroxyl ions are emitted into the opening.

25. The apparatus of clause 1, further comprising a control system having:
a humidity sensor to monitor a humidity discharged by the apparatus,
a temperature sensor to monitor a temperature of the hydroxyl ions, and
a flow sensor to monitor a flow rate of the hydroxyl ions.

26. The apparatus of clause 25, wherein the control system adjusts an atomizer based on the humidity as measured by the humidity sensor.

27. The apparatus of clause 26, wherein the control system adjusts the atomizer to maintain a humidity within a range of 40% to 60%.

28. The apparatus of clause 26, wherein the control system adjusts the atomizer to maintain a humidity within a range of 57% to 59%.

29. The apparatus of clause 26, wherein the control system adjusts the atomizer to maintain a humidity below 60%.

30. The apparatus of clause 25, wherein the control system adjusts an atomizer based on the temperature of the hydroxyl ions as measured by the temperature sensor.

31. The apparatus of clause 25, wherein the control system adjusts an atomizer based on the flow rate of the hydroxyl ions as measured by the flow sensor.

32. The apparatus of clause 25, wherein the control system adjusts an atomizer based on combination of at least two of the humidity as measured by the humidity sensor, the temperature as measured by the temperature sensor, and the flow rate as measured by the flow sensor.

33. The apparatus of clause 25, wherein the control system adjusts a light of the hydroxyl ion generator based on the temperature as measured by the temperature sensor.

34. The apparatus of clause 25, wherein the control system adjusts a light of the hydroxyl ion generator based on the flow rate as measured by the flow sensor.

35. The apparatus of clause 25, wherein the control system adjusts a light of the hydroxyl ion generator based on a combination of the temperature as measured by the temperature sensor and the flow rate as measured by the flow sensor.

36. The apparatus of clause 25, wherein the control system controls the hydroxyl ion generator based on the temperature as measured by the temperature sensor.

37. The apparatus of clause 25, wherein the control system controls the hydroxyl ion generator based on the flow rate as measured by the flow sensor.

38. The apparatus of clause 25, wherein the control system controls the hydroxyl ion generator based on combination of the temperature as measured by the temperature sensor and the flow rate as measured by the flow sensor.

39. The apparatus of clause 25, wherein the control system includes a wireless transceiver, wherein the apparatus is remotely controllable by a remote device through the wireless transceiver.

40. The apparatus of clause 25, wherein the humidity sensor includes:
an inlet humidity sensor positioned at an air intake of the portal, and
an outlet humidity sensor positioned at the at least one opening of the portal.

41. The apparatus of clause 25, wherein the temperature sensor includes:
an inlet temperature sensor positioned at an air intake of the portal, and
an outlet temperature sensor positioned at the at least one opening of the portal.

42. The apparatus of clause 25, wherein the flow rate sensor includes:
an inlet flow rate sensor positioned at an air intake of the portal, and
an outlet flow rate sensor positioned at the at least one opening of the portal.

43. The apparatus of clause 1, further comprising an ozone sensor, wherein the portal is inactivated if a predetermined level of ozone is detected in the portal.

44. The apparatus of clause 1, further comprising a carbon dioxide sensor, wherein the portal is inactivated if a predetermined level of carbon dioxide is detected in the portal.

45. The apparatus of clause 1, further comprising a carbon monoxide sensor, wherein the portal is inactivated if a predetermined level of carbon monoxide is detected in the portal.

46. The apparatus of clause 1, wherein the panel of the portal is positioned in a doorway so that the hydroxyl ions are emitted into the doorway.

47. The apparatus of clause 1, wherein the panel of the portal is positioned on a floor so that the hydroxyl ions are emitted upward from the floor.

48. The apparatus of clause 1, wherein the panel of the portal is embedded in a floor so that the hydroxyl ions are emitted upward from the floor.

49. The apparatus of clause 1, wherein the hydroxyl ions are free of ozone.

50. The apparatus of clause 1, wherein the at least one opening includes a plurality of openings.

51. The apparatus of clause 50, wherein the plurality of openings are formed in an array.

52. The apparatus of clause 1, further comprising a filter to purify the hydroxyl ions emitted.

53. The apparatus of clause 52, wherein the filter includes an approximately 0.3 micron filter.

54. The apparatus of clause 1, wherein the panel includes a plurality of panels coupled together.

55. A method to disinfect a surface comprising the steps:
passing an object through the apparatus of any of clauses 1 through 62, wherein the object is subjected to hydroxyl ions in an amount sufficient to reduce or eliminate one or more pathogens on the surface of the object.

56. The method of clause 55, wherein the humidification system provides at least 60% humidity for use with the UV light and titanium oxide to generate the hydroxyl ions.

57. The method of clause 55, wherein the object is an inanimate object or is a living being.

58. The method of clause 55, wherein the living being is an individual or a mammal.

59. The method of clause 55, wherein the object is subjected to hydroxyl ions through the portal for a period of 1 second to 30 seconds.

60. The method of clause 55, wherein the hydroxyl concentration is at a level that reduces or eliminates one or more pathogens upon exposure to the hydroxyl ions.

61. The method of clause 55, further comprising adjusting an atomizer of the apparatus based on the humidity as measured by a humidity sensor.

62. The method of clause 55, further comprising adjusting an atomizer of the apparatus based on a temperature as measured by a temperature sensor.

63. The method of clause 55, further comprising adjusting an atomizer of the apparatus based on a flow rate as measured by a flow sensor.

64. The method of clause 55, further comprising adjusting an atomizer of the apparatus based on combination of at least two of a humidity as measured by a humidity sensor, a temperature measured by a temperature sensor, and a flow rate as measured by a flow sensor.

65. The method of clause 55, further comprising adjusting a light of the apparatus based on a temperature as measured by a temperature sensor.

66. The method of clause 55, further comprising adjusting a light of the apparatus based on a flow rate as measured by a flow sensor.

67. The method of clause 55, further comprising adjusting a light of the apparatus based on a combination of a temperature as measured by a temperature sensor and a flow rate as measured by a flow sensor.

68. The method of clause 55, further comprising adjusting a hydroxyl ion generator based on a temperature as measured by a temperature sensor.

69. The method of clause 55, further comprising adjusting a hydroxyl ion generator based on a flow rate as measured by a flow sensor.

70. The method of clause 55, further comprising adjusting a hydroxyl ion generator based on combination of a temperature as measured by a temperature sensor and a flow rate as measured by a flow sensor.

71. The method of clause 55, further comprising maintaining a humidity within a range of 40% to 60%.

72. The method of clause 55, further comprising maintaining a humidity within a range of 57% to 59%.

73. The method of clause 55, further comprising maintaining a humidity below 60%.

74. The method of clause 55, further comprising inactivating the apparatus if a predetermined level of ozone is detected.

75. The method of clause 55, further comprising inactivating the apparatus if a predetermined level of carbon dioxide is detected.

76. The method of clause 55, further comprising inactivating the apparatus if a predetermined level of carbon monoxide is detected.

77. An apparatus comprising:
a directional airflow apparatus; and
a hydroxyl ion generator in communication with the directional airflow apparatus, wherein hydroxyl ions can be transmitted through the directional airflow apparatus to emit the hydroxyl ions into the surrounding area.

78. The apparatus of clause 77, wherein the directional airflow apparatus is a conduit, ductwork, piping, or tubing.

79. The apparatus of clause 77, wherein the directional airflow apparatus is configured to disperse the hydroxyl ions into an enclosed space.

80. The apparatus of clause 77, further comprising at least one fan in communication with the hydroxyl ion generator.

81. The apparatus of clause 77, wherein the hydroxyl ion generator comprises a perforated mesh coated with titanium dioxide, a water source, a humidification system and a UV light generator that provides a spectrum of light from about 320 nm to about 385 nm.

82. The apparatus of clause 81, wherein the water source is a continuous water source.

83. The apparatus of clause 81, wherein the water source includes water stored in a container.

84. The apparatus of clause 83, further comprising an attached float to regulate the level of water in the container.

85. The apparatus of clause 81, wherein the humidification system is an ultrasonic humidification system.

86. The apparatus of any of clauses 77 through 85, further comprising a water filter in communication with the water source and the humidification system.

87. The apparatus of clause 77, further comprising a control system having:
a humidity sensor to monitor a humidity discharged by the apparatus,
a temperature sensor to monitor a temperature of the hydroxyl ions, and
a flow sensor to monitor a flow rate of the hydroxyl ions.

88. The apparatus of clause 87, wherein the control system adjusts an atomizer based on the humidity as measured by the humidity sensor.

89. The apparatus of clause 88, wherein the control system adjusts the atomizer to maintain a humidity within a range of 40% to 60%.

90. The apparatus of clause 88, wherein the control system adjusts the atomizer
to maintain a humidity within a range of 57% to 59%.

91. The apparatus of clause 88, wherein the control system adjusts the atomizer to maintain a humidity below 60%.

92. The apparatus of clause 87, wherein the control system adjusts an atomizer based on the temperature as measured by the temperature sensor.

93. The apparatus of clause 87, wherein the control system adjusts an atomizer based on the flow rate as measured by the flow sensor.

94. The apparatus of clause 87, wherein the control system adjusts an atomizer based on combination of at least two of the humidity as measured by the humidity sensor, the temperature as measured by the temperature sensor, and the flow rate as measured by the flow sensor.

95. The apparatus of clause 87, wherein the control system adjusts a light of the hydroxyl ion generator based on the temperature as measured by the temperature sensor.

96. The apparatus of clause 87, wherein the control system adjusts a light of the hydroxyl ion generator based on the flow rate as measured by the flow sensor.

97. The apparatus of clause 87, wherein the control system adjusts a light of the hydroxyl ion generator based on combination of the temperature as measured by the temperature sensor and the flow rate as measured by the flow sensor.

98. The apparatus of clause 87, wherein the control system controls the hydroxyl ion generator based on the temperature as measured by the temperature sensor.

99. The apparatus of clause 87, wherein the control system controls the hydroxyl ion generator based on the flow rate as measured by the flow sensor.

100. The apparatus of clause 87, wherein the control system controls the hydroxyl ion generator based on combination of the temperature as measured by the temperature sensor and the flow rate as measured by the flow sensor.

101. The apparatus of clause 77, further comprising a plurality of directional airflow apparatuses configured to emit the hydroxyl ions into the surrounding area.

102. The apparatus of clause 101, wherein the plurality of directional airflow apparatuses includes:
a master directional airflow apparatus, and
at least one slave directional airflow apparatus that is controlled by the master directional airflow apparatus.

103. The apparatus of clause 102, wherein each of the master directional airflow apparatus and the at least one slave directional airflow apparatus includes a wireless transceiver, wherein the each of the master directional airflow apparatus and the at least one slave directional airflow apparatus communicate through the wireless transceiver.

104. The apparatus of clause 102, wherein the each of the master directional airflow apparatus and the at least one slave directional airflow apparatus are remotely controllable by a remote device through the wireless transceiver.

105. The apparatus of clause 101, wherein:
the master directional airflow apparatus includes a control system, and
each of the plurality of directional airflow apparatuses includes a humidity sensor to monitor a humidity emitted from the respective directional airflow apparatus, a temperature sensor to monitor a temperature of the hydroxyl ions emitted from the respective directional airflow apparatus, and a flow sensor to monitor a flow rate of the hydroxyl ions emitted from the respective directional airflow apparatus.

106. The apparatus of clause 105, wherein the control system adjusts an atomizer of at least one of the plurality of directional airflow apparatuses based on the humidity as measured by the humidity sensor of at least one of the plurality of directional airflow apparatuses.

107. The apparatus of clause 106, wherein the control system adjusts the atomizer of at least one of the plurality of directional airflow apparatuses to maintain a humidity within the surrounding area within a range of 40% to 60%.

108. The apparatus of clause 106, wherein the control system adjusts the atomizer of at least one of plurality of directional airflow apparatuses to maintain a humidity within the surrounding area within a range of 57% to 59%.

109. The apparatus of clause 106, wherein the control system adjusts the atomizer of at least one of the plurality of directional airflow apparatuses to maintain a humidity within the surrounding area below 60%.

110. The apparatus of clause 105, wherein the control system adjusts an atomizer of at least one of the plurality of directional airflow apparatuses based on the temperature as measured by the temperature sensor of at least one of the plurality of directional airflow apparatuses.

111. The apparatus of clause 106, wherein the control system adjusts an atomizer of at least one of the plurality of directional airflow apparatuses based on the flow rate as measured by the flow sensor of at least one of the plurality of directional airflow apparatuses.

112. The apparatus of clause 105, wherein the control system adjusts an atomizer of at least one of the plurality of directional airflow apparatuses based on combination of at least two of the humidity as measured by the humidity sensor of at least one of the plurality of directional airflow apparatuses, the temperature as measured by the temperature sensor of at least one of the plurality of directional airflow apparatuses, and the flow rate as measured by the flow sensor airflow of at least one of the plurality of directional airflow apparatuses.

113. The apparatus of clause 105, wherein the control system adjusts a light of at least one of the plurality of directional airflow apparatuses based on the temperature as measured by the temperature sensor of at least one of the plurality of directional airflow apparatuses.

114. The apparatus of clause 105, wherein the control system adjusts a light of at least one of the plurality of directional airflow apparatuses based on the flow rate as measured by the flow sensor of at least one of the plurality of directional airflow apparatuses.

115. The apparatus of clause 105, wherein the control system adjusts a light of at least one of the plurality of directional airflow apparatuses based on combination of the temperature as measured by the temperature sensor of at least one of the plurality of directional airflow apparatuses and the flow rate as measured by the flow sensor airflow of at least one of the plurality of directional airflow apparatuses.

116. The apparatus of clause 105, wherein the control system controls the hydroxyl ion generator of at least one of the plurality of directional airflow apparatuses based on the temperature as measured by the temperature sensor of at least one of the plurality of directional airflow apparatuses.

117. The apparatus of clause 105, wherein the control system controls the hydroxyl ion generator of at least one of the plurality of directional airflow apparatuses based on the flow rate as measured by the flow sensor of at least one of the plurality of directional airflow apparatuses.

118. The apparatus of clause 105, wherein the control system controls the hydroxyl ion generator of at least one of the plurality of directional airflow apparatuses based on combination of the temperature as measured by the temperature sensor of at least one of the plurality of directional airflow apparatuses and the flow rate as measured by the flow sensor of at least one of the plurality of directional airflow apparatuses.

119. The apparatus of clause 87, wherein the control system includes a wireless transceiver, wherein the apparatus is remotely controllable by a remote device through the wireless transceiver.

120. The apparatus of clause 87, wherein the humidity sensor includes:
an inlet humidity sensor positioned at an air intake of the directional airflow apparatus, and
an outlet humidity sensor positioned at an outlet of the directional airflow apparatus.

121. The apparatus of clause 87, wherein the temperature sensor includes:
an inlet temperature sensor positioned at an air intake of the directional airflow apparatus, and
an outlet temperature sensor positioned at an outlet of the directional airflow apparatus.

122. The apparatus of clause 87, wherein the flow rate sensor includes:
an inlet flow rate sensor positioned at an air intake of the directional airflow apparatus, and
an outlet flow rate sensor positioned at an outlet of the directional airflow apparatus.

123. The apparatus of clause 77, further comprising an ozone sensor, wherein the directional airflow apparatus is inactivated if a predetermined level of ozone is detected by the ozone sensor.

124. The apparatus of clause 77, further comprising a carbon dioxide sensor, wherein the directional airflow apparatus is inactivated if a predetermined level of carbon dioxide is detected by the carbon dioxide sensor.

125. The apparatus of clause 77, further comprising a carbon monoxide sensor, wherein the directional airflow apparatus is inactivated if a predetermined level of carbon monoxide is detected by the carbon monoxide sensor.

126. The apparatus of clause 77, wherein the directional airflow apparatus is positioned on a wall.

127. The apparatus of clause 77, wherein the directional airflow apparatus is positioned in a wall.

128. The apparatus of clause 77, wherein the directional airflow apparatus is positioned on a floor.

129. The apparatus of clause 77, wherein the directional airflow apparatus is positioned in a floor.

130. The apparatus of clause 77, wherein the directional airflow apparatus is positioned on a ceiling.

131. The apparatus of clause 77, wherein the directional airflow apparatus is positioned in a ceiling.

132. The apparatus of clause 77, wherein the hydroxyl ions is free of ozone.

133. The apparatus of clause 77, further comprising a filter to purify the hydroxyl ions emitted from the directional airflow apparatus.

134. The apparatus of clause 133, wherein the filter includes an approximately 0.3 micron filter.

135. The apparatus of clause 77, further comprising:
a water channel;
at least one atomizer positioned in the water channel and configured to atomize water in the water channel; and
a light emitting diode, wherein atomized water passes over the light emitting diode.

136. The apparatus of clause 135, wherein the at least one atomizer seals the water channel.

137. A method to disinfect a surface or air space comprising the steps:
treating an enclosed area with the apparatus of any of clauses 77 through 136, wherein the enclosed area is subjected to hydroxyl ions in an amount sufficient to reduce or eliminate one or more pathogens throughout the air in the enclosed area and/or on the surface of objects within the enclosed area.

138. The method of clause 137, wherein the humidification system provides at least 60% humidity for use with the UV light and titanium oxide to generate the hydroxyl ions.

139. The method of clause 137, wherein the hydroxyl concentration is at a level that reduces or eliminates one or more pathogens upon exposure to the hydroxyl ions.

140. The method of clause 137, wherein the percentage reduction of one or more pathogens is between about 40% and 100%, e.g. 44% to about 92%, upon exposure to hydroxyl ion from between about 15 minutes to about 90 minutes.

141. The method of clause 137, further comprising adjusting an atomizer based on a humidity as measured by a humidity sensor.

142. The method of clause 137, further comprising adjusting an atomizer based on a temperature as measured by a temperature sensor.

143. The method of clause 137, further comprising adjusting an atomizer based on a flow rate as measured by a flow sensor.

144. The method of clause 137, further comprising adjusting an atomizer based on combination of at least two of a humidity as measured by a humidity sensor, a temperature as measured by a temperature sensor, and a flow rate as measured by a flow sensor.

145. The method of clause 137, further comprising adjusting a light based on a temperature as measured by a temperature sensor.

146. The method of clause 137, further comprising adjusting a light based on a flow rate as measured by a flow sensor.

147. The method of clause 137, further comprising adjusting a light based on combination of a temperature as measured by a temperature sensor and a flow rate as measured by a flow sensor.

148. The method of clause 137, further comprising adjusting a hydroxyl ion generator based on a temperature as measured by a temperature sensor.

149. The method of clause 137, further comprising adjusting a hydroxyl ion generator based on a flow rate as measured by a flow sensor.

150. The method of clause 137, further comprising adjusting a hydroxyl ion generator based on combination of a temperature as measured by a temperature sensor and a flow rate as measured by a flow sensor.

151. The method of clause 137, further comprising maintaining a humidity within a range of 40% to 60%.

152. The method of clause 137, further comprising maintaining a within a range of 57% to 59%.

153. The method of clause 137, further comprising maintaining a humidity below 60%.

154. The method of clause 137, further comprising controlling at least one slave directional airflow apparatus with a master directional airflow apparatus.

155. The method of clause 137, further comprising inactivating the apparatus if a predetermined level of ozone is detected.

156. The method of clause 137, further comprising inactivating the apparatus if a predetermined level of carbon dioxide is detected.

157. The method of clause 137, further comprising inactivating the apparatus if a predetermined level of carbon monoxide is detected.

158. A system for producing hydroxyl ions in an amount sufficient to reduce or eliminate one or more pathogens, the system including:
at least one apparatus having a hydroxyl ion generator to produce hydroxyl ions and at least one fan to move the hydroxyl ions,
a control system to control the at least one apparatus,
a humidity sensor to monitor a humidity discharged from the at least one apparatus,
a temperature sensor to monitor a temperature of the hydroxyl ions, and
a flow sensor to monitor a flow rate of the hydroxyl ions.

159. The system of clause 158, wherein at least one of the humidity sensor, the temperature sensor, and the flow sensor is located at the apparatus.

160. The system of clause 158, wherein at least one of the humidity sensor, the temperature sensor, and the flow sensor is located remotely from the apparatus.

161. The system of clause 158, wherein at least one of the humidity sensor, the temperature sensor, and the flow sensor is wirelessly coupled to the control system.

162. The system of clause 158, wherein the control system adjusts an atomizer based on the humidity as measured by the humidity sensor.

163. The system of clause 162, wherein the control system adjusts the atomizer to maintain a humidity within a range of 40% to 60%.

164. The system of clause 162, wherein the control system adjusts the atomizer to maintain a humidity within a range of 57% to 59%.

163. The system of clause 162, wherein the control system adjusts the atomizer to maintain a humidity below 60%.

164. The system of clause 158, wherein the control system adjusts an atomizer based on the temperature as measured by the temperature sensor.

165. The system of clause 158, wherein the control system adjusts an atomizer based on the flow rate as measured by the flow sensor.

166. The system of clause 158, wherein the control system adjusts an atomizer based on combination of at least two of the humidity as measured by the humidity sensor, the temperature as measured by the temperature sensor, and the flow rate as measured by the flow sensor.

167. The system of clause 158, wherein the control system adjusts a light of the hydroxyl ion generator based on the temperature as measured by the temperature sensor.

168. The system of clause 158, wherein the control system adjusts a light of the hydroxyl ion generator based on the flow rate as measured by the flow sensor.

169. The system of clause 158, wherein the control system adjusts a light of the hydroxyl ion generator based on combination of the temperature as measured by the temperature sensor and the flow rate as measured by the flow sensor.

170. The system of clause 158, wherein the control system controls the hydroxyl ion generator based on the temperature as measured by the temperature sensor.

171. The system of clause 158, wherein the control system controls the hydroxyl ion generator based on the flow rate as measured by the flow sensor.

172. The system of clause 158, wherein the control system controls the hydroxyl ion generator based on combination of the temperature as measured by the temperature sensor and the flow rate as measured by the flow sensor.

173. The system of clause 158, further comprising a plurality of apparatuses configured to emit the hydroxyl ions.

174. The system of clause 173, wherein the plurality of apparatuses includes:
a master apparatus, and
at least one slave apparatus that is controlled by the master apparatus.

175. The system of clause 158 further comprising an ozone sensor, wherein the directional airflow apparatus is inactivated if a predetermined level of ozone is detected by the ozone sensor.

176. The system of clause 158, further comprising a carbon dioxide sensor, wherein the directional airflow apparatus is inactivated if a predetermined level of carbon dioxide is detected by the carbon dioxide sensor.

177. The system of clause 158, further comprising a carbon monoxide sensor, wherein the directional airflow apparatus is inactivated if a predetermined level of carbon monoxide is detected by the carbon monoxide sensor.

178. A method to reduce or eliminate a pathogen on a surface or in the air comprising the step of subjecting air to hydroxyl ions generated by an apparatus or system of any of clauses 1 through 54, 77 through 136 or 158 through 177, wherein the presence of the pathogen is reduced or eliminated.

179. The method of clause 178, wherein the pathogen is an allergen, pollen, protozoa, fungi, molds, viruses, bacteria or mixtures thereof.

180. A method to decompose or degrade a pathogen on a surface or in the air comprising the step of subjecting air to hydroxyl ions generated by an apparatus or system of any of clauses 1 through 54, 77 through 136 or 158 through 177, wherein the presence of the pathogen is decomposed or degraded.

181. The method of clause 180, wherein the pathogen is an allergen, pollen, protozoa, fungi, molds, viruses, bacteria or mixtures thereof.

182. A method to reduce or eliminate one or more volatile organic components or particulates in air comprising the step of subjecting air with one or more volatile organic component or particulates to hydroxyl ions generated by an apparatus of any of clauses 1 through 54, 77 through 136 or 158 through 177, wherein the presence of the one or more volatile organic component or particulates are reduced or eliminated from the air.

183. The apparatus of clause 77, wherein the directional airflow apparatus and the hydroxyl ion generator are portable.

184. The apparatus of clause 183, further comprising a portable cart, wherein the directional airflow apparatus and the hydroxyl ion generator are positioned on the portable cart.

185. The apparatus of clause 183, further comprising a portable water source.

186. The apparatus of clause 185, wherein the portable water source is refillable.

187. The apparatus of clause 185, further comprising a water channel in fluid communication with the portable water source, wherein the water channel includes:
a solenoid valve configured to operate between an open state and a closed state, wherein, in the open state, water flows from the portable water source to the water channel, and, in the closed state, a flow of water from the portable water source to the water channel is restricted, and
an atomizer configured to atomize water in the water channel.

188. The apparatus of clause 77, further comprising a water channel in fluid communication with a water source, wherein the water channel includes:
a solenoid valve configured to operate between an open state and a closed state, wherein, in the open state, water flows from the water source to the water channel, and, in the closed state, a flow of water from the water source to the water channel is restricted, and
an atomizer configured to atomize water in the water channel.

189. The apparatus of clauses 1 and 77, further comprising an atomizer system configured to generate water droplets that are directed into the hydroxyl ion generator.

190. The apparatus of clause 189, wherein the atomizer system comprises:
a housing defining a cavity, wherein the cavity is configured to retain water, and
an atomizer positioned in the cavity and configured to be submerged by the water in the cavity, wherein the atomizer generates the water droplets.

191. The apparatus of clause 190, wherein the atomizer system further comprises a water inlet configured to couple to a water source to dispense water into the cavity.

192. The apparatus of clause 190, wherein the atomizer system further comprises:
a channel extending from the cavity, wherein water flows from the cavity into the channel, and
a sensor configured to detect water in the channel,
wherein a flow of water into the cavity is controlled based on a water level in the channel detected by the sensor.

193. The apparatus of clause 192, wherein the channel extends approximately 90 degrees from the cavity.

194. The apparatus of clause 190, wherein the atomizer system further comprises:
an air intake configured to direct air into the cavity, and
an air outlet configured to direct air containing the water droplets out of the cavity.

195. The apparatus of clause 194, wherein the air outlet is positioned adjacent a fan and the fan is configured to direct the air containing the water droplets into the hydroxyl ion generator.

196. The apparatus of clause 189, further comprising a fan positioned adjacent the atomizer system, wherein the fan is configured to direct the water droplets from the atomizer system into the hydroxyl ion generator.

197. The system of clause 158, wherein the at least one apparatus further comprises an atomizer system configured to generate water droplets that are directed into the hydroxyl ion generator.

198. The system of clause 197, wherein the atomizer system comprises:
a housing defining a cavity, wherein the cavity is configured to retain water, and
an atomizer positioned in the cavity and configured to be submerged by the water in the cavity, wherein the atomizer generates the water droplets.

199. The system of clause 198, wherein the atomizer system further comprises a water inlet configured to couple to a water source to dispense water into the cavity.

200. The system of clause 198, wherein the atomizer system further comprises:
a channel extending from the cavity, wherein water flows from the cavity into the channel, and
a sensor configured to detect water in the channel,
wherein a flow of water into the cavity is controlled based on a water level in the channel detected by the sensor.

201. The system of clause 200, wherein the channel extends approximately 90 degrees from the cavity.

202. The system of clause 198, wherein the atomizer system further comprises:
an air intake configured to direct air into the cavity, and
an air outlet configured to direct air containing the water droplets out of the cavity.

203. The system of clause 202, wherein the air outlet is positioned adjacent a fan and the fan is configured to direct the air containing the water droplets into the hydroxyl ion generator.

204. The system of clause 197, further comprising a fan positioned adjacent the atomizer system, wherein the fan is configured to direct the water droplets from the atomizer system into the hydroxyl ion generator.

205. An apparatus comprising:
a hydroxyl ion generator configured to emit hydroxyl ions into a surrounding area,
an atomizer system configured to generate water droplets from water retained in a cavity, wherein an atomizer is positioned in the cavity and configured to be submerged by the water in the cavity, wherein the atomizer generates the water droplets, and
a fan positioned adjacent the atomizer system, wherein the fan is configured to direct the water droplets from the atomizer system into the hydroxyl ion generator.

206. The apparatus of clause 205, wherein the atomizer system further comprises a water inlet configured to couple to a water source to dispense water into the cavity.

207. The apparatus of clause 205, wherein the atomizer system further comprises:
a channel extending from the cavity, wherein water flows from the cavity into the channel, and
a sensor configured to detect water in the channel,
wherein a flow of water into the cavity is controlled based on a water level in the channel detected by the sensor.

208. The apparatus of clause 207, wherein the channel extends approximately 90 degrees from the cavity.

209. The apparatus of clause 205, wherein the atomizer system further comprises:
an air intake configured to direct air into the cavity, and
an air outlet configured to direct air containing the water droplets out of the cavity.

210. The apparatus of clause 209, wherein the air outlet is positioned adjacent the fan.

What is claimed is:

1. An apparatus comprising:
a hydroxyl ion generator configured to emit hydroxyl ions into a surrounding area,
an atomizer system configured to generate water droplets from water retained in a cavity, wherein an atomizer is positioned in the cavity and configured to be submerged by the water in the cavity, wherein the atomizer generates the water droplets, and
a fan positioned adjacent the atomizer system, wherein the fan is configured to direct the water droplets from the atomizer system into the hydroxyl ion generator.

2. The apparatus of claim 1 further comprising a portal comprising at least one opening, wherein the hydroxyl ions are transmitted through the at least one opening to emit the hydroxyl ions into an area formed by the portal.

3. The apparatus of claim 1, wherein the hydroxyl ion generator comprises:
a perforated mesh coated with titanium dioxide and configured to collect the water droplets, and
a UV light generator that directs a spectrum of light from about 320 nm to about 385 nm onto the perforated mesh.

4. The apparatus of claim 1, wherein the water source is at least one of a continuous water source and a container.

5. The apparatus of claim 1, further comprising a control system having:
a humidity sensor to monitor a humidity discharged by the apparatus,
a temperature sensor to monitor a temperature of the hydroxyl ions, and
a flow sensor to monitor a flow rate of the hydroxyl ions.

6. The apparatus of claim 5, wherein the control system adjusts the atomizer based on at least one of the humidity as measured by the humidity sensor, the temperature as measured by the temperature sensor, and the flow rate as measured by the flow sensor.

7. The apparatus of claim 5, wherein the control system adjusts the hydroxyl ion generator based on at least one of the humidity as measured by the humidity sensor, the temperature as measured by the temperature sensor, and the flow rate as measured by the flow sensor.

8. The apparatus of claim 5, further comprising a UV light generator that directs a spectrum of light from about 320 nm to about 385 nm onto the water droplets, wherein the control system adjusts the atomizer based on at least one of the temperature as measured by the temperature sensor, and the flow rate as measured by the flow sensor.

9. The apparatus of claim 1, further comprising at least one of:
an ozone sensor, wherein the apparatus is inactivated if a predetermined level of ozone is detected in an area around the apparatus,
a carbon dioxide sensor, wherein the apparatus is inactivated if a predetermined level of carbon dioxide is detected in the area around the apparatus, and
a carbon monoxide sensor, wherein the apparatus is inactivated if a predetermined level of carbon monoxide is detected in the area around the apparatus.

10. The apparatus of claim 1, wherein a hydroxyl concentration of the hydroxyl ions is at a level that reduces or eliminates one or more pathogens upon exposure to the hydroxyl ions.

11. The apparatus of claim 1, further comprising a plurality of apparatuses including a master apparatus and at least one slave apparatus that is controlled by the master apparatus.

12. The apparatus of claim 11, wherein each of the master apparatus and the at least one slave apparatus include a wireless transceiver, wherein the master apparatus and the at least one slave apparatus communicate through the wireless transceiver.

13. The apparatus of claim 11, wherein the master apparatus and the at least one slave apparatus are remotely controllable by a remote device through the wireless transceiver.

14. The apparatus of claim 1, wherein the atomizer system provides at least 60% humidity to the hydroxyl ion generator.

15. The apparatus of claim 1, wherein the atomizer system further comprises a water inlet configured to couple to a water source to dispense water into the cavity.

16. The apparatus of claim 1, wherein the atomizer system further comprises a sensor configured to detect an amount of water in the cavity, wherein a flow of water into the cavity is controlled based on the amount of water detected in the cavity.

17. The apparatus of claim 1, wherein the atomizer system further comprises:
an air intake configured to direct air into the cavity, and
an air outlet configured to direct air containing the water droplets out of the cavity.

18. The apparatus of claim 17, wherein the air outlet is positioned adjacent the fan, wherein the fan is configured to direct the air containing the water droplets into the hydroxyl ion generator.

19. The apparatus of claim 1, further comprising a portable cart, wherein the apparatus is positioned on the portable cart.

20. The apparatus of claim 1, wherein the apparatus is installed in at least one of a floor, a wall, and a ceiling.
